(19) United States
(12) Patent Application Publication
(10) Pub. No.: US 2013/0090548 A1
(43) Pub. Date: Apr. 11, 2013

(54) AUTOMATED RENAL EVALUATION SYSTEMS AND METHODS USING MRI IMAGE DATA

(71) Applicant: Wake Forest University Health Sciences, Winston-Salem, NC (US)

(72) Inventors: Craig Alan Hamilton, Lewisville, NC (US); William Gregory Hundley, Winston-Salem, NC (US); Matthew Stevens Edwards, Winston-Salem, NC (US); Michael Vito Rocco, Greensboro, NC (US)

(73) Assignee: Wake Forest University Health Sciences, Winston-Salem, NC (US)

(21) Appl. No.: 13/648,656

(22) Filed: Oct. 10, 2012

Related U.S. Application Data
(60) Provisional application No. 61/545,431, filed on Oct. 10, 2011.

(51) Int. Cl. A61B 5/00; A61M 5/14; A61B 5/01; A61B 5/055; A61B 5/145

(52) U.S. Cl. CPC A61B 5/004; A61B 5/055; A61B 5/14542; A61B 5/4872; A61B 5/015; A61M 5/14
USPC 600/411; 600/419; 600/412

(57) ABSTRACT

Renal screening systems include a circuit configured to electronically analyze MRI image data of a subject to evaluate renal function and generate a renal-risk report for a plurality of different therapeutic agents based on renal responses to test doses of each of the agents.

### RELATED APPLICATIONS

This application claims the benefit of and priority to U.S. Provisional Patent Application Ser. No. 61/545,431, filed Oct. 10, 2011, the contents of which are hereby incorporated by reference as if recited in full herein.

### STATEMENT OF GOVERNMENT SUPPORT

This invention was made with government support under Grant Nos. R41AG030248 and R42AG030248 from the National Institutes of Health. The United States government has certain rights in the invention.

### FIELD OF THE INVENTION

The present invention is related to evaluation of renal disorders, diseases or injuries or therapy impact on kidneys using MRI image data.

### BACKGROUND OF THE INVENTION

Atherosclerotic renal artery stenosis (aRAS) is an increasingly recognized cause of chronic kidney disease (CKD) and end stage renal disease. aRAS is also strongly associated with increased risks for cardiac events and mortality, with these effects likely due in large part to associated hypertension and kidney dysfunction. Unfortunately, the pathophysiology of aRAS-associated CKD is poorly understood. The current estimated prevalence of aRAS among Americans over the age of 65 is 7%, or more than 3.5 million individuals. RA-RT (including stent placement and surgical bypass) is used to treat aRAS in hopes of reducing the observed kidney-related and cardiovascular morbidity and mortality. Currently, over 45,000 RA-RT procedures are performed each year in the U.S. with a cost of over $500 million. Unfortunately, even with the best current patient selection measures, only about 20-50% of individuals treated with RA-RT experience significant improvement in their kidney function. Renal function improvement has been demonstrated to be the most important predictor of subsequent overall and dialysis-free survival. The observed variability in kidney function response to RA-RT is due to an incomplete understanding of the pathophysiology of aRAS-associated CKD and the current inability to measure the functional reserve, or ‘retrievability’ of kidney tissue distal to an aRAS lesion.

### SUMMARY OF EMBODIMENTS OF THE INVENTION

Embodiments of the invention provide systems, methods and computer program products that can provide one or more of: (a) an automated analysis of renal MRI images; and/or (b) a workstation with a display that can provide a user a suite of rendered kidney tissue maps and/or MR images that show oxygenation, blood flow, perfusion and/or other parameters of interest associated with kidney function.

The systems can provide a more efficient and improved diagnostic assessment tool over conventional renal assessment systems which may employ more manual analysis and less kidney functional data.

Embodiments of the invention electronically evaluate and/or electronically generate a suite of different MRI renal images and tissue maps to assess renal tissue oxygenation, vascular oxygenation, flow measurements in the renal artery, blood perfusion in the kidney as well as structural angiograms.

Embodiments of the invention can provide systems, circuits and methods that carry out an automated renal screening analysis that correlates kidney function to different potential therapies for treating kidney disease or injury and/or for treating other conditions with drug therapies that may have an unintended or undesired impact on kidney function (e.g., diabetes medicines, blood pressure medicines, heart disease medicines and the like) to allow a more informed selection of a drug therapy based on identification of the risk that kidney function may be undesirably affected by a particular drug therapy. The evaluation can automatically determine and show in one or more tissue maps whether oxygenation, perfusion or blood flow is negatively impacted by one or more drugs.

The screening can be carried out while administering a series of different test doses of drugs, typically having a relatively short half-life, while obtaining MRI image data and correlating the respective administered drugs to an associated set of MRI images, then automatically analyzing the images to generate a report with an indication of which, if any, of the drugs may present a risk of injury, dysfunction or otherwise induce a negative reaction or response and/or which is likely to be a safer choice for preserving (or even potentially improving) renal function and the like.

The screening/automated analysis can be carried out rapidly, as a “rapid” screening evaluation, typically within about 24 hours of cessation of a patient MRI scan session, more typically within about two hours and in some embodiments within about 1 hour or less.

Embodiments of the invention have broad applicability in nephrology. One, and typically all of, renal blood flow, renal blood perfusion, renal tissue and vascular oxygenation and renal functional reserve can be evaluated by automated analysis using MRI image data. The analysis can be used to screen those patients more likely to benefit from RV or to select an appropriate therapy, e.g., medicine or surgery.

The analysis can evaluate or identify those not likely to benefit from RV, identify patients likely to benefit from drug therapy to delay dialysis, or tailor a medicine to a patient for better medical intervention choices for certain conditions.

The analysis can assist in tailoring patient-specific therapy of antihypertensive and heart failure medications in patients, including those with CKD, to preserve renal function or inhibit further damage or injury.

Embodiments of the invention are directed to renal evaluation systems. The systems include a circuit comprising at least one processor configured to: (i) segment cortical and medullary regions of different MRI kidney image slices of a respective patient into defined sub-segments for volume analysis and associate borders of the defined sub-segments with a respective color; (ii) assess oxygenation and perfusion in the defined sub-segments before and after one or more agents are administered to a respective patient; and (iii) generate a color coded image of abdominal fat adjacent a respective kidney of a patient. The systems can also include at least one display in communication with the circuit configured to display the color coded image of abdominal fat of a patient and at least one image slice of a segmented kidney with defined sub-segments with color borders.

The defined sub-segments can include a total kidney volume, a medulla volume, and a renal sinus volume. The circuit can be configured to analyze each kidney image slice having a slice thickness between about 3 mm to about 20 mm to (i) calculate a cortical volume as equal to total kidney volume minus medulla volume and to (ii) calculate a medullary volume as equal to the medulla volume minus the renal sinus volume. The circuit can be configured to evaluate whether blood flow changes in response to administered agents preserve or alter renal cortex to medullary volume ratios.

The circuit can be configured to calculate blood flow and percent stenosis of at least one renal artery.

The circuit can be configured to identify whether the patient is likely to benefit or likely not to benefit from a medical or procedure therapy (for example, a pharmaceutical regimen and/or revascularization therapy).

The circuit can be configured to analyze at least one of tissue oxygenation, vascular oxygenation, renal arterial blood flow by comparing base line MRI image data and MRI images obtained after administration of a therapy delivered proximate in time to an MRI scan session used to obtain post-therapy MRI image data of the kidney or kidneys.

The circuit can be further configured to generate color and/or heat spectrum tissue maps of a patient's kidney or kidneys, the tissue maps illustrating the kidney or kidneys with associated pixel values defined based at least in part on at least one of (i) a ratio of T1 and T2*; (ii) a weighted combination of T1 and T2*, or (iii) a T2* difference map and a T1 difference map using corresponding pixels associated with respective T1 and T2* MR images obtained before and after administration of an agent, the T2* difference map visually illustrates vascular oxygenation in color scale and the T1 difference map visually illustrates tissue oxygenation in color scale.

Other embodiments of the invention are directed to therapeutic renal screening systems. The systems include a circuit configured to electronically analyze MRI images of at least one kidney of a subject to evaluate renal function based on renal responses to test doses of each of a plurality of different defined therapeutic agents, wherein the circuit evaluates at least one of (a) change in tissue oxygenation, (b) change in vascular oxygenation, and (c) renal artery blood flow rates to evaluate the renal responses. The circuit generates a renal risk report for the different therapeutic agents based on the patient's renal response to the test doses of each of the agents.

The different agents are for treating a condition other than kidney disease.

The systems can include a workstation with a display in communication with the circuit, the circuit configured to analyze the MRI images, generate the renal risk report and transmit the renal risk report to the workstation display within about 24 hours after a respective subject's MR scan session used to obtain the MRI images.

The circuits can be configured to generate a rapid screening analysis with one or more associated reports, the analysis being carried out and the one or more reports transmitted to a clinician within about 2 hours after the subject's MR scan session.

The circuits can be in communication with an infusion pump, a plurality of test doses of the different therapeutic agents configured for IV administration and a control circuit for directing the serial delivery of the test doses. The therapeutic agents can be administered as oral agents during therapeutic use and the test doses can be substantially pharmaceutically equivalent formulations of the therapeutic agents configured for IV administration.

The systems can include a display in communication with the circuit and an electronic library module in communication with the circuit, the electronic library module comprising lists of different therapeutic agents correlated to different defined conditions, and wherein a user can select a condition from the defined conditions and the circuit presents associated different therapeutic agents to the display.

The library of different conditions include at least two of the following conditions: diabetes, COPD, asthma, heart failure, heart disease, chemotherapy, infection, and high blood pressure.

The test doses can be provided in a kit of test vials or pouches.

The risk reports can include a color risk evaluation for each of the different therapeutic agents ranging from high to low risk of kidney complications or undesired kidney response, including a first color for low risk, a second color for a moderate risk, and third color for a high risk.

The risk report can include a numerical risk index evaluation for each of the different therapeutic agents ranging from high to low risk of kidney complications or undesired kidney response, on a numerical index from 1-10, with 1 being a low risk and 10 being a high risk.

The risk report can include a color risk evaluation and/or a numerical risk index from 1-10 for each of the different therapeutic agents ranging from high to low risk of kidney complications or undesired kidney response, including “green” and a number “1” for low risk, “yellow” and a number “5” for a moderate risk, and “red” and a number “10” for a high risk on a numerical index from 1-10.

In some embodiments, the systems, methods and computer program products can evaluate the ability of new compounds or drugs that may be effective (or not) for treating CKD to preserve renal function or for treating other conditions without impairing kidney function or causing kidney injury.

In some embodiments, the systems, methods and computer program products can evaluate the effect of an oral or intravenous agent, typically one used in an intensive care setting, on the preservation of renal function and/or on the likelihood of recovery of acute renal failure of a patient. Thus, for example, medical interventions for diabetes, high blood pressure, chronic heart failure, heart disease and the like can be carried out with more information regarding which agent is suitable for a particular patient due to the evaluated pharmacologic agent's affect on the kidney(s).

Some embodiments of the invention can employ at least one, and typically a series of, defined pharmacologic agent in a formulation having a short half-life (e.g., liquid form for an IV drip) and acquiring MRI image data that is used to assess a kidney's response to the agent(s). This evaluation can be carried out relatively rapidly as a “rapid drug compatibility screening” to allow a clinician to be able to select an appropriate medication within 24 hours, typically within about 30 minutes to about 2 hours, from the start or end of an MRI scan session of a respective patient.

Typically, some if not most or all of the automated analysis can be carried out during an MRI scan session as different MRI scans are obtained, using multiple MRI scans and automated image analysis.

A parametric color-coded renal map can be generated using T1, T2* and perfusion pixel/voxel data.

A suite of MR renal evaluations or tests (angiogram, flow T1, T2*, perfusion) can be provided with a UI for ease of use and patient evaluation.

In some embodiments, an entire study (non-contrast arteriogram, renal blood flow measures (at rest and after diuretic) and renal tissue oxygenation (before and after diuretic) of a patient can be obtained in about 1 hour, and in some embodiments, in under one hour, such as about 30 minutes or less, measured from a start or an end of an MRI scanner session of a respective patient.

In some embodiments, simultaneous visualization of renal arteries on a display with measurement of renal blood flow and determination of kidney oxygenation in a single examination can be generated without the need for contrast agents.

It is contemplated that embodiments of the invention can evaluate the pathophysiology of the CKD associated with aRAS and a potential solution to the problem of optimal patient selection. Blood Oxygen Level Dependent (BOLD) data assessed from R2* acquisitions (1/T2*) during MRI can be used to measure baseline levels of kidney tissue oxygenation and changes in these tissue oxygen levels after administration of a loop diuretic to suppress the metabolic demands of solute reabsorption. These data can be acquired safely without using intravenous contrast materials or ionizing radiation and may provide essential information regarding the pathologic changes in the kidney associated with aRAS and the retrievability of kidney function distal to an aRAS lesion.

Embodiments of the invention can evaluate renal tissue oxygen levels, and changes in those levels with diuretic administration. The systems can determine 1) whether those renal oxygen levels are low, e.g., lower in kidneys with aRAS (when compared to kidneys without aRAS); and 2) identify those kidneys with aRAS exhibiting significantly increased function post-RA-RT and/or significantly lower pre-RA-RT tissue oxygen levels, and significant changes in those levels with diuretic administration, when compared with kidneys with aRAS exhibiting unchanged or worsened function post RA-RT.

As will be appreciated by those of skill in the art in light of the present disclosure, embodiments of the present invention may be provided as methods, systems and/or computer program products. Claims presented as method claims can be carried out programmatically via one or more digital signal processors.

It is noted that any one or more aspects or features described with respect to one embodiment, may be incorporated in a different embodiment although not specifically described relative thereto. That is, all embodiments and/or features of any embodiment can be combined in any way and/or combination. Applicant reserves the right to change any originally filed claim or file any new claim accordingly, including the right to be able to amend any originally filed claim to depend from and/or incorporate any feature of any other claim although not originally claimed in that manner These and other objects and/or aspects of the present invention are explained in detail in the specification set forth below.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 is a block diagram of an MRI system according to embodiments of the present invention;

FIG. 2 is a block diagram of a data processing system according to embodiments of the present invention;

FIG. 3 is a block diagram of a data processing system according to embodiments of the present invention;

FIG. 4 is an example of a T2* map obtained using a T2* decay from images at multiple TEs fit (exponential function) using a decay curve of signal over time for images obtained at different time according to embodiments of the present invention. Cortical and medullary ROIs can be manually identified (traced).

FIGS. 5A and 5B are T1 color maps (shown in grey scale) with the pre-agent T1 color map shown in FIG. 5A and the post-agent T1 map shown in FIG. 5B according to embodiments of the present invention.

FIGS. 6A and 6B are T2* color maps (shown in grey scale) with pre-agent map shown in FIG. 6A and the post-agent map shown in FIG. 6B (using the same agent used to generate FIG. 5B) according to embodiments of the present invention.

FIG. 7 is a coronal ASL image of a different patient, which illustrates the differences in the image types.

FIGS. 8A-8C are exemplary color-coded tissue maps (in gray scale) that can be simultaneously or selectively shown on a display associated with a workstation according to embodiments of the present invention. FIG. 8A is a T1 map. FIG. 8B is a T2 map.

FIG. 8C is a weighted-sum map of the maps of T1 and T2 according to embodiments of the present invention.

FIG. 9 are axial and lower coronal 3D angiograms of right and left renal arteries with visual indicia (e.g., arrows) showing near total occlusion of the left artery and about 50% stenosis of the right artery (the more severe occlusion can be shown in a different color or opacity for visual emphasis) according to embodiments of the present invention.

FIG. 10 is a graph of flow (ml/min) versus time (ms) of flow measurements over a cardiac cycle illustrating pre- and post-agent administration flow rates according to embodiments of the present invention.

FIGS. 11A and 11B are graphs of manual versus automated renal artery blood flow (ml/min) and stress/rest changes in flow (FIG. 11B) according to embodiments of the present invention (in use, the automated analysis may be shown without the manual flow calculation as the manual one is shown for comparison as to accuracy).

FIG. 12 is a renal image showing four different measurements of the kidney that can be shown simultaneously or concurrently on a display for ease of diagnosis according to embodiments of the present invention.

FIG. 13 is a flow chart of exemplary renal tissue mapping for renal viability assessment according to embodiments of the present invention.

FIG. 14 is a block diagram of automated analysis of renal MR images according to embodiments of the present invention.

FIG. 15A is a schematic illustration of an MRI evaluation system that uses MRI data according to embodiments of the present invention.

FIG. 15B is an exemplary prophetic section view of a kidney that shows different tissue parameters obtained using MRI data according to embodiments of the present invention.

FIG. 16 is a schematic illustration of an MRI-based renal evaluation system according to embodiments of the present invention.

FIG. 17 is a schematic illustration of an MRI-based renal evaluation system according to other embodiments of the present invention.

FIG. 18A is a schematic illustration of a drug dispensing assembly for use in a renal evaluation system according to embodiments of the present invention.

FIG. 18B is a schematic illustration of a multi-drug reservoir block for use in a renal evaluation system according to embodiments of the present invention.

FIGS. 19A-19D are schematic illustrations of exemplary renal evaluation reports according to embodiments of the present invention.

FIG. 20 is a schematic illustration of another exemplary screen renal evaluation report according to embodiments of the present invention.

FIG. 21 is a schematic illustration of a kit or package of test doses of different therapeutic agents for use in a screening evaluation of a subject according to embodiments of the present invention.

FIG. 22 is a schematic illustration of an electronic library of different conditions undergoing therapy and a correlated list of alternative therapeutic agents according to some embodiments of the present invention.

FIGS. 23 and 24 are flow charts of exemplary operations that can be carried out according to embodiments of the present invention.

FIGS. 25A and 26A are arterial spin labeling images of respective patient kidneys.

FIGS. 25B and 25C are pre and post furosemide T2* images of the kidney shown in FIG. 25A.

FIGS. 26B and 26C are pre and post furosemide T2* images of the kidney shown in FIG. 26A.

FIG. 27A is an axial MRI image of at a second lumbar vertebral body.

FIG. 27B is a color coded MRI image of different abdominal fat compartments according to embodiments of the present invention.

FIG. 28A is a screen shot of multiple overlapping images of kidneys identifying segments of the kidney volume with different color borders or perimeters according to embodiments of the present invention.

FIG. 28B is an example of a segmentation of a kidney for volume analyses with borders in different colors representing different kidney volumes that can be repeated for each slice (an exemplary slice thickness ST of 10 mm).

FIGS. 29A-29F are images with the segmented kidney volumes shown with color borders as those volumes change over time in response to different drug challenges according to embodiments of the present invention.

FIG. 30 is a flow chart of automated image processing steps that can be carried out according to embodiments of the present invention.

FIGS. 31A and 31D are images of different patient left kidneys. FIGS. 31B and 31C are T2* (BOLD) pre and post furosemide therapy images of the kidney of the patient in FIG. 31A. FIGS. 31E and 31F are pre and post furosemide therapy T2* (BOLD) images of a patient with the left kidney shown in FIG. 31D on chronic medication of furosemide pre and post administration of a challenge or temporally administered image dose according to embodiments of the present invention.

FIGS. 32A and 32B are color coded BOLD pre and post lasix T2* MRI images of kidneys with associated image parameter (e.g., intensity) values to the right thereof according to embodiments of the present invention.

FIGS. 33A and 33B are phase contrast images showing the middle right renal artery. FIG. 33C is a graph of flow (ml/s) versus time (ms) with a summary of related parameters that can be automatically calculated using the image data according to embodiments of the present invention.

The figures may include prophetic examples of screen shots of visualizations and the like and do not necessarily represent actual screen shots of a surgical system/display.

### DESCRIPTION OF EMBODIMENTS OF THE INVENTION

The present invention now will be described more fully hereinafter with reference to the accompanying drawings, in which embodiments of the invention are shown. However, this invention should not be construed as limited to the embodiments set forth herein. Rather, these embodiments are provided so that this disclosure will be thorough and complete, and will fully convey the scope of the invention to those skilled in the art. Like numbers refer to like elements throughout. As used herein the term “and/or” includes any and all combinations of one or more of the associated listed items. Broken lines illustrate optional features or operations unless specified otherwise. In the claims, the claimed methods are not limited to the order of any steps recited unless so stated thereat.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

The term “about” means that the stated number can vary between +/−20% of the stated value.

It will be understood that, although the terms first, second, etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein. Well-known functions or constructions may not be described in detail for brevity and/or clarity.

The term “interactive” refers to a device and/or algorithm that can respond to user input to provide an output. The user input can be using touch gestures, pull down menus, mouse or screen touch instruments. The user can define a ROI (region of interest) in an image using a UI to allow for better registration.

As is known to those of skill in the art, the phrase “drawing a region of interest in air,” does not literally mean “in air,” but rather that the line or curve is drawn outside the body (and/or heart) in the image to obtain a corresponding background of noise data that can be used to adjust voxel intensity data.

The actual visualization shown on a display, such as that associated with a clinician workstation, can be shown on a screen or display so that the map of the anatomical structure is in a flat 2-D and/or in 2-D what appears to be 3-D volumetric images with data representing features or tissue characteristics with different visual characteristics such as with differing intensity, opacity, color, texture and the like. Alternatively, actual projection 3-D images or cines may also be shown on a display. A 4-D map can either illustrate a renal artery with blood flow or show additional information over a 3-D anatomic model of the contours of the kidney or portions thereof. The term “kidney” can include adjacent vasculature.

The term “workstation” refers to a computer having a display or screen associated with a clinician, such as a doctor, nurse or other medical personnel or, for research use, with a researcher.

The term “color scale” refers to using color to visually represent differences in a measure of a property of a pixel/voxel, such as intensity, T2, T2*, T1 or ratios or weighted values of same, with similar colors representing similar values. Different values can have different colors. Small differences may be indicated by a graduated scale of the same color. The term “color coded” refers to a defined color for a defined (common) tissue (e.g., specific fat volume), image parameter or region.

The term “map” is used interchangeably with the term “model” and refers to a volumetric rendering or visualization of an image of a patient's target anatomy (e.g., kidney or portions thereof). The map can be rendered or generated showing one or more selected tissue parameters, conditions, or behaviors of kidney tissue using MR image data, e.g., the tissue map can be a rendered partial or global anatomical map of the kidney or kidneys of a patient using calculated pixel values from one or more different MRI image types such as, for example, T1, T2* or a ratio of T1/T2*, a difference map of one or both and/or a weighted, combined tissue map. The map can be configured to be electronically rotated, sectioned or otherwise manipulated for ease of view to allow a clinician to interrogate features thereof. The map can be visualized in a manner that illustrates relative degrees or measures of a tissue characteristic(s) of interest, typically in different colors, opacities and/or intensities.

In some embodiments, some selected MRI-derived tissue data from the tissue map or the map(s) themselves can be selectively turned on and off (on a display) or faded. Several different tissue maps may be merged, combined, or shown as a composite map. Different maps may be shown overlying and aligned with one another. Thus, the visualizations can use different volumetric tissue maps, shown separately, overlaid on each other and/or integrated as a composite (weighted and/or summed pixel values) or superimposed maps. The terms “fade” and “faded” refer to making the so-called feature and/or voxel characteristic less visually dominant in a visualization by dimming the intensity, color and/or opacity relative to other features, voxel characteristics or parameters in the visualization.

In some embodiments of the present invention, the measure of intensity, where used, may be average, median and/or mean intensity of the pixels of respective images.

In some embodiments or aspects, a difference image of corresponding pixels or voxels from different images may be used to generate a difference image or portion of an image. In some embodiments, weighted measures of pixels from different images may be used to generate an image. In some embodiments ratios of two MRI tissue characteristics can be used such as, for example, T1/T2, T1/T2* or the inverses thereof.

The term “parametric image” refers to an image that illustrates a relative or absolute measure of a defined a tissue characteristic or parameter or parameters, such as oxygenation, perfusion, blood flow (or combinations thereof) of the kidney on a pixel by pixel basis, e.g., the pixel value can be mapped to a location using a coordinate system. Different ones of these values can be combined from different MRI images using the defined location.

In some embodiments, various different RF excitation pulse sequences can be used to obtain MRI image data with desired renal tissue parameter data associated with perfusion, tissue or vascular oxygenation, blood flow, or other desired functions. The pulse sequences can be used with or without contrast agents, and with or without “challenge” or other drug or agent administration. Typically, the MRI image data is obtained without contrast agents and with administration of one or more defined drug or agent.

In some embodiments, quantitative T2* measurements of vascular oxygenation in the kidneys can be obtained using BOLD imaging sequences and T2 mapping. The T2* measurements can provide a sequence of images whose intensities vary in relation to the T2* of the kidney, which is an MRI tissue characteristic dependent on the oxygen present in the blood in the capillaries of the renal tissue (vascular oxygenation).

In some embodiments, T1 measurements can be used to assess tissue oxygenation in the kidneys using T1 mapping. T1 is influenced by the amount of oxygen present in the renal tissue itself (tissue oxygenation). T1 image data may also or alternatively be used to assess if renal fibrosis is present.

In some embodiments, arterial spin labeling (ASL) can be used to assess renal blood perfusion. ASL is a non-contrast technique using a patient's blood as an endogenous contrast agent to measure blood perfusion, an indicator of functionality of the renal tissue.

Table 1 provides examples of some optional (exemplary) image parameters for T2* maps, ASL, T1 maps, phase contrast measures of blood flow in the renal artery and the non-contrast angiogram that can be used. As is well known to those of skill in the art these are general guidelines/parameters only. The parameters may be modified across different scanner platforms and/or manufacturers. As a result, the parameters in Table 1 are intended as a “rough” guide as to what can be used to acquire the images as is well known to those of skill in the art. Although DWI (diffusion weighted image) parameters are not shown, those of skill in the art will understand the parameters used to obtain these type of images.

TABLE 1

| | | | | Phase | Non-Contrast |
|---|---|---|---|---|---|
| Description | T2* map | ASL | T1 Map | Contrast | Angiogram |
| Sequence Type | 2D | 2D Steady- | Steady State | 2D Phase | 3D Steady |
| | Gradient- | State Free | Free | Contrast | State Free |
| | Echo | Precession | Precession | Gradient Echo | Procession |
| FOV | 440 mm | 440 mm | 440 mm | 320 mm | 340 mm |
| Phase FOV | 90.6% | 90.6% | 90.6% | 75% | 71.7% |
| TR | 200 | 5000 | 1000 | 42.85 | 1400 |
| TE | 0.93 | 1.74 | 0.87 | 2.61 | 1.72 |
| Flip Angle | 18 | 60 | 35 | 15 | 90 |
| NEX (Averages) | 1 | 1 | 1 | 1 | 1 |
| Concatenations | — | 1 | 1 | — | 1 |
| (Slices) | | | | | |
| Bandwidth | 1953 | 977 | 1028 | 491 | 783 |
| (Hz/pixel) | | | | | |
| Gating | — | — | — | ecg | Respiratory |
| | | | | | Navigator |
| Slice Thickness | 10 mm | 10 mm | 10 mm | 5 mm | 0.91 mm |
| Segments | 1 | 1 | 58 | 4 | 37 |
| Matrix | 116 × 128 | 232 × 256 | 116 × 128 | 144 × 192 | 198 × 304 |

The perfusion information can be combined with the other measures in a color-coded representation of the kidney where the color can indicate tissue viability.

Diffusion weighted imaging (DWI) can also be used to provide renal image data.

The images can include each or combinations of image data from two or more of T1, T2 or T2* renal images.

Stress ratios of one or more of the different tissue maps can be electronically generated.

A structural angiogram can be provided as a 3D set of data with the ability to zoom, rotate, slice and reformat. Software (electronic) calipers can be provided to measure lumen diameter or area at points along a renal artery for quantification of renal stenosis severity. Embodiments of the invention can automatically identify those patients having severe stenosis, e.g., about 75% or greater occlusion.

Flow measurements can be automatically determined using images where pixel values reflect velocity of blood flow in the renal artery.

The measurements can be automated using a circuit such as a computer program, at least one processor, and/or software for automatic lumen segmentation and extraction of parameters of interest such as mean flow over a cardiac cycle, peak velocity and flow volume. Ratios before and after drug or agent administration may be used to provide flow reserve measures which indicate vascular functional reserve.

Selected absolute or relative values of each pixel in regions of interest in one or more images can be evaluated, e.g., electronically evaluated to determine the value for each pixel correlated to a respective location.

Changes over time in a particular patient may be electronically evaluated or shown on a display to illustrate or emphasize relative differences in a patient's own image data, or a patient's image data can be compared to a norm or defined standard to visually identify, emphasize and/or electronically assess “high”, “low” or other abnormal measure of function.

In some embodiments, pre- and post-drug or post-agent (during or post-administration) image scans can be obtained. The pre- and post-drug/agent images can be registered and difference maps can be computed to assess for changes. In some embodiments, the pre- and post-drug/agent images can be selectively displayed or automatically displayed adjacently or as one or more cines of time-elapsed kidney oxygenation and/or perfusion changes on a display associated with a workstation.

Tissue oxygenation and vascular oxygenation color maps of one or both kidneys (or image slices thereof) can be displayed side by side or one can be selectively or automatically faded into another by allowing a user to alter a desired view using a GUI.

The drug can be a therapeutic drug to evaluate whether a patient might benefit from the therapy. The drug or agent can be used in a chemical “challenge” to try to force a functional change in the kidney(s), e.g., a diuretic such as furosemide or LASIX. The term “drug” includes pharmaceuticals. The term “agent” includes any biocompatible substance used to force or vary a body function. The administration of the drug or agent can be used to tailor patient specific therapies (drug type and/or dose) and/or to test the ability of potential drugs to perform one or more of: (i) not cause kidney injury or damage (ii) preserve renal function or (iii) recover renal function.

A user can select to illustrate side-by-side images of different patient renal images on a screen or display associated with a clinician workstation. This includes static and cines of MR renal maps and/or images. The cines can show dynamic tissue perfusion, oxygenation, blood flow and the like over a defined timeline. The timeline can be any desired timeline, which may be shown in an accelerated format. The timeline can be, for example, between 1 minute to 1 hour, such as 5 minutes, 10 minutes, and any time increment therebetween. The cines can be generated to illustrate functional changes pre- and post-drug administration and/or over time. The cines can be based on a difference model or map of pre- and post-drug administration. Alternatively or additionally, a user can select to display the images or cines side by side, registered to be “in synch”.

The systems, methods, circuits and/or computer program products can be used during and/or post-scan as a data processing system to automatically electronically analyze patient data for renal evaluations.

Alternatively or additionally, the systems, methods or computer program products can be used while a patient is in an MRI scanner undergoing evaluation to provide rapid or substantially real-time diagnostic data.

As will be appreciated by one of skill in the art, the present invention may be embodied as methods, systems, or computer program products. Accordingly, the present invention may take the form of an entirely hardware embodiment, an entirely software embodiment or an embodiment combining software and hardware aspects all generally referred to herein as a “circuit” or “module.” Furthermore, the present invention may take the form of a computer program product on a computer-usable storage medium having computer-usable program code embodied in the medium. Any suitable computer readable medium may be utilized including hard disks, CD-ROMs, optical storage devices, a transmission media such as those supporting the Internet or an intranet, or magnetic storage devices.

Computer program code for carrying out operations of the present invention may be written in an object oriented programming language such as Java®, Smalltalk or C++. However, the computer program code for carrying out operations of the present invention may also be written in conventional procedural programming languages, such as the “C” programming language. The program code may execute entirely on a user's computer, entirely or partly on an MR Scanner, partly on the user's computer, as a stand-alone software package, partly on the user's computer and partly on a remote computer or entirely on the remote computer. In the latter scenario, the remote computer may be connected to the user's computer through a local area network (LAN) or a wide area network (WAN), or the connection may be made to an external computer (for example, through the Internet using an Internet Service Provider) using HIPPA appropriate firewalls and data exchange protocols. Furthermore, the user's computer, the remote computer, or both, may be integrated into other systems, such as an MRI Scanner, an HIS (Hospital Information System), and/or a PACs system.

The present invention is described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer-readable memory that can direct a computer or other programmable data processing apparatus to function in a particular manner, such that the instructions stored in the computer-readable memory produce an article of manufacture including instruction means which implement the function/act specified in the flowchart and/or block diagram block or blocks.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions/acts specified in the flowchart and/or block diagram block or blocks.

While embodiments of the present invention may be particularly useful in identifying those patients that are likely to benefit from revascularization as well as those that are not likely to see a target improvement, embodiments of the present invention may also be utilized in evaluating patients for other kidney issues, including those that may be identified early to delay any requirement of dialysis, diabetic changes, in drug discovery programs, clinical trials and/or diagnostic environments using data from the detection.

To compare serial acquisitions of MRI images and related pixel and/or voxel data, alignment of the slices for the images (aligning the image slices from different acquisitions) can be important to reliably detect intensity changes in pixels/voxels in different images of a patient and/or to be able to discard less relevant neighborhoods of pixels/voxels that might skew the intensity values (and hence the analysis) of a certain region or regions of the kidney being evaluated or interrogated.

As noted above, certain embodiments of the present invention may provide for contrast/intensity analysis without the administration of a contrast agent. For example, using blood oxygen level dependent (BOLD) renal imaging.

BOLD MRI renal tissue oxygen data and kidney-specific glomerular filtration rates in individuals and kidneys with and without aRAS can be used to identify tissue hypoxia in aRAS-associated CKD.

BOLD MRI renal tissue oxygen data in kidneys with aRAS and subsequent kidney-specific function response following RA-RT.

Changes in BOLD MRI renal tissue oxygen data and kidney-specific glomerular filtration rate can be evaluated between about 2-4 weeks post-RA-RT to assess hypoxia correction in the success or failure of RA-RT to improve kidney function.

Functional Renal MRI can measure a number of physiologic processes within the kidney in a noninvasive manner and can be performed without the use of gadolinium contrast, iodine based contrast or ionizing radiation. Therefore, kidneys can be imaged regardless of the current level of kidney function, including patients who are oliguric or anuric.

MRI-derived measures of oxygenation and regional blood flow can be provided that are not available with other imaging techniques and to detect differences in pathophysiology that may be relevant in determining the likelihood of recovery from AKI.

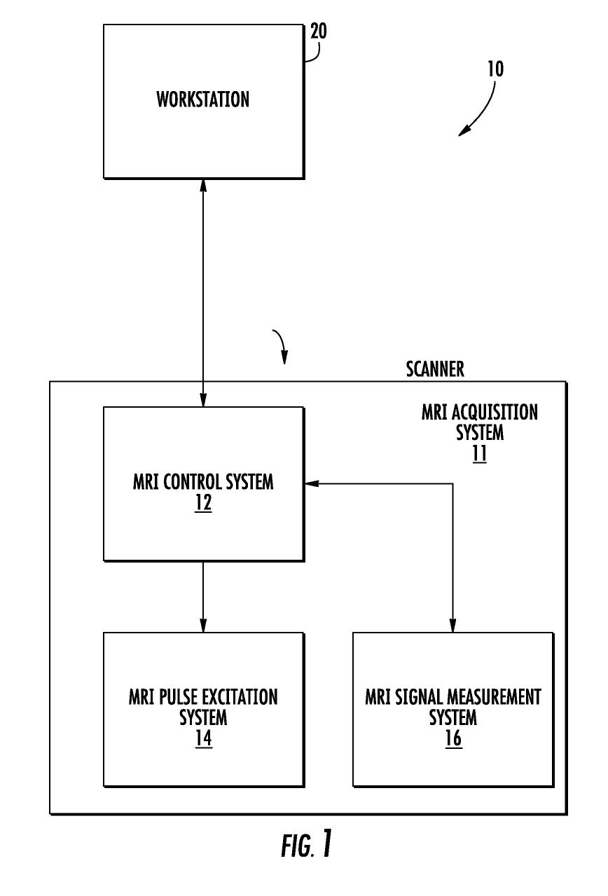

An exemplary system 10 according to embodiments of the present invention is illustrated in FIG. 1. As seen in FIG. 1, MRI analysis system 10 is in communication with or includes an MRI acquisition system 11 that may include an MRI control system circuit 12, an MRI pulse excitation system circuit 14 and an MRI signal measurement system circuit 16. The MRI control system circuit 12 controls operations of the MRI acquisition system 11 to obtain and provide MRI images during a cardiac cycle or portions thereof of a patient. The MRI control system circuit 12 may also assemble and transmit the acquired images to a workstation 20 or other such data processing system for further analysis and/or display on an associated display 20D. The workstation 20 may be in an MRI suite or may be remote from the MRI suite. The MRI pulse excitation system circuit 14 and the MRI signal measurement system circuit 16 are controlled to acquire MRI signals that may provide MRI images of the heart of a patient.

Conventional MRI systems, such as those provided by General Electric Medical Systems, Siemens, Philips, Varian, Bruker, Marconi, Hitachi and Toshiba may be utilized to provide the desired MRI images and/or MR image data (typically collected after administration of a contrast agent). The MRI systems (also known as MR Scanners) can be any suitable magnetic field strength, such as, for example, about 1.5 T or 2.0 T, and may be higher field systems, such as above 2.0 T to about 10.0 T. The magnets can be open or closed bore magnets.

While an exemplary intensity analysis/MRI system is illustrated in FIG. 1 and described herein with a particular division of functions and/or operations, as will be appreciated by those of skill in the art, other divisions of functions and/or operations may be utilized while still benefiting from the teachings of the present invention. For example, the MRI control system circuit 12 could be combined with either the MRI pulse excitation system circuit 14 or the MRI signal measurement system circuit 16. Thus, the present invention should not be construed as limited to a particular architecture or division of MRI functions/operations but is intended to cover any architecture or division of functions/operations capable of carrying out the operations described herein.

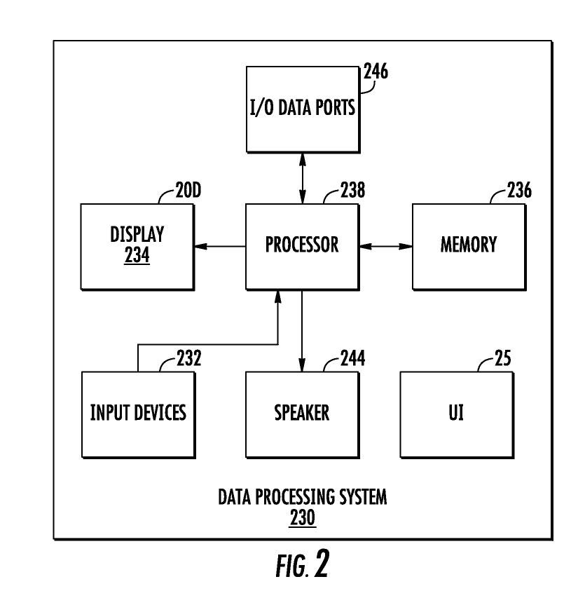

FIG. 2 illustrates an exemplary embodiment of a data processing system 230 suitable for providing a workstation 20 and/or MRI control system circuit 12 in accordance with embodiments of the present invention. The MRI control system circuit 12 can be incorporated into the MR Scanner control cabinet in the control room of an MRI suite. The magnet can be held in the magnet room with RF shielding as is well known. The data processing system 230 typically includes input device(s) 232 such as a keyboard or keypad, a display 234 (also referred to as “20D”), and a memory 236 that communicate with a processor 238. The data processing system 230 may further include a speaker 244, and an I/O data port(s) 246 that also communicate with the processor 238. The I/O data ports 246 can be used to transfer information between the data processing system 230 and another computer system or a network such as an intranet or the Internet and may include a PACS. PACS (PICTURE ARCHIVING AND COMMUNICATION SYSTEM) is a system that receives images from imaging modalities, stores the data in archives, and distributes the data to clinicians for viewing (and can refer to sub portions of these systems).

These components may be conventional components such as those used in many conventional data processing systems that may be configured to operate as described herein. The module or circuit can be provide using one or more servers that can be provided using cloud computing which includes the provision of computational resources on demand via a computer network. The resources can be embodied as various infrastructure services (e.g. computer, storage, etc.) as well as applications, databases, file services, email, etc. In the traditional model of computing, both data and software are typically fully contained on the user's computer; in cloud computing, the user's computer may contain little software or data (perhaps an operating system and/or web browser), and may serve as little more than a display terminal for processes occurring on a network of external computers. A cloud computing service (or an aggregation of multiple cloud resources) may be generally referred to as the “Cloud”. Cloud storage may include a model of networked computer data storage where data is stored on multiple virtual servers, rather than being hosted on one or more dedicated servers. Data transfer can be encrypted and can be done via the Internet using any appropriate firewalls to comply with industry or regulatory standards such as HIPAA. The term “HIPAA” refers to the United States laws defined by the Health Insurance Portability and Accountability Act. The patient data can include an accession number or identifier, gender, age and image data as well as segmented abdominal fat compartment data.

FIG. 3 is a block diagram of embodiments of data processing systems that illustrates systems, methods, and computer program products in accordance with embodiments of the present invention. The processor 238 communicates with the memory 236 via an address/data bus 348. The processor 238 can be any commercially available or custom microprocessor. The memory 236 is representative of the overall hierarchy of memory devices containing the software and data used to implement the functionality of the data processing system 230. The memory 236 can include, but is not limited to, the following types of devices: cache, ROM, PROM, EPROM, EEPROM, flash memory, SRAM, and DRAM.

As shown in FIG. 3, the memory 236 may include several categories of software and/or data used in the data processing system 230: the operating system 352; the application programs 354; the input/output (I/O) device drivers 358; and the data 356. As will be appreciated by those of skill in the art, the operating system 352 may be any operating system suitable for use with a data processing system, such as OS/2, AIX or System390 from International Business Machines Corporation, Armonk, N.Y., Windows95, Windows98, Windows2000, WindowsNT or WindowsXP from Microsoft Corporation, Redmond, Wash., Unix or Linux. The operating systems may be configured to support a TCP/IP-based or other such network communication protocol connection. The I/O device drivers 358 typically include software routines accessed through the operating system 352 by the application programs 354 to communicate with devices such as the I/O data port(s) 246 and certain memory 236 components. The application programs 354 are illustrative of the programs that implement the various features of the data processing system 230 and preferably include at least one application that supports operations according to embodiments of the present invention. Finally, the data 356 represents the static and dynamic data used by the application programs 354, the operating system 352, the I/O device drivers 358, and other software programs that may reside in the memory 236.

As is further seen in FIG. 3, the application programs 354 may include a renal (MRI image data) analysis application 360. The renal analysis application 360 may carry out the operations described herein for evaluating images to detect changes in a tissue property that may be associated with kidney function and/or viability. The data portion 356 of memory 236, as shown in the embodiments of FIG. 3, may include image data 362, such as MRI image data from one or more images.

While the present invention is illustrated, for example, with reference to the renal analysis application 360 being an application program in FIG. 3, as will be appreciated by those of skill in the art, other configurations may also be utilized while still benefiting from the teachings of the present invention. For example, the renal analysis application 360 may also be incorporated into the operating system 352, the I/O device drivers 358 or other such logical division of the data processing system 230. Thus, the present invention should not be construed as limited to the configuration of FIG. 3 but is intended to encompass any configuration capable of carrying out the operations described herein.

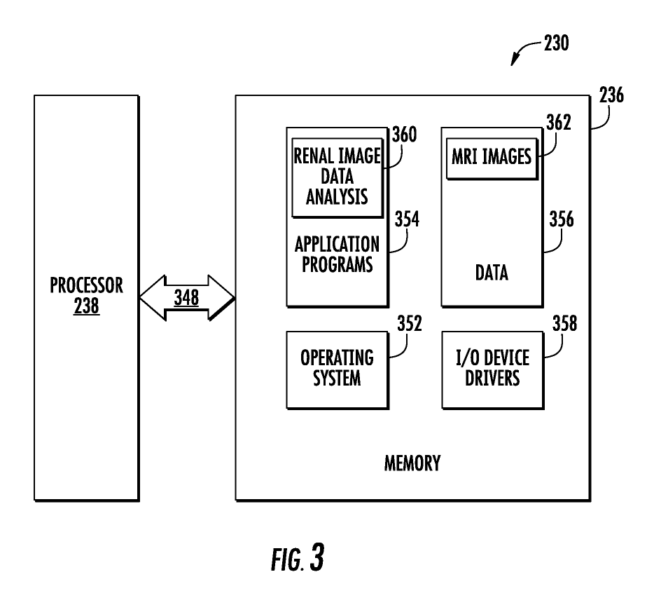

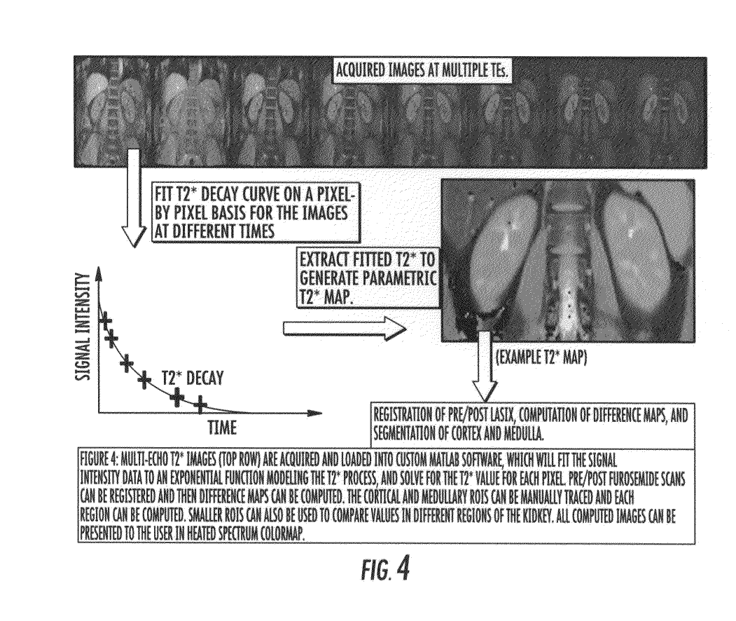

FIG. 4 is an example of a T2* map obtained using a T2* decay from images at multiple TEs fit (exponential function) using a decay curve of signal intensity data over time for images obtained at different time according to embodiments of the present invention. Cortical and medullary ROIs can be manually identified (traced). MR images can be acquired at multiple TEs (top row); the T2* decay curve (exponential function modeling the T2* process) can be fit on a pixel by pixel basis for the images at different times. The fitted T2* data can be extracted to generate a parametric T2* map (right side). Pre and post furosemide scans can be registered and difference maps generated. The cortex and medulla regions of interest (ROIs) can be segmented electronically using a GUI input that allows a user to manually trace the regions. Smaller ROIs can also be used to compare values in different regions of the kidney. The maps or computed images can be presented in a heated spectrum color map or other color-coded map.

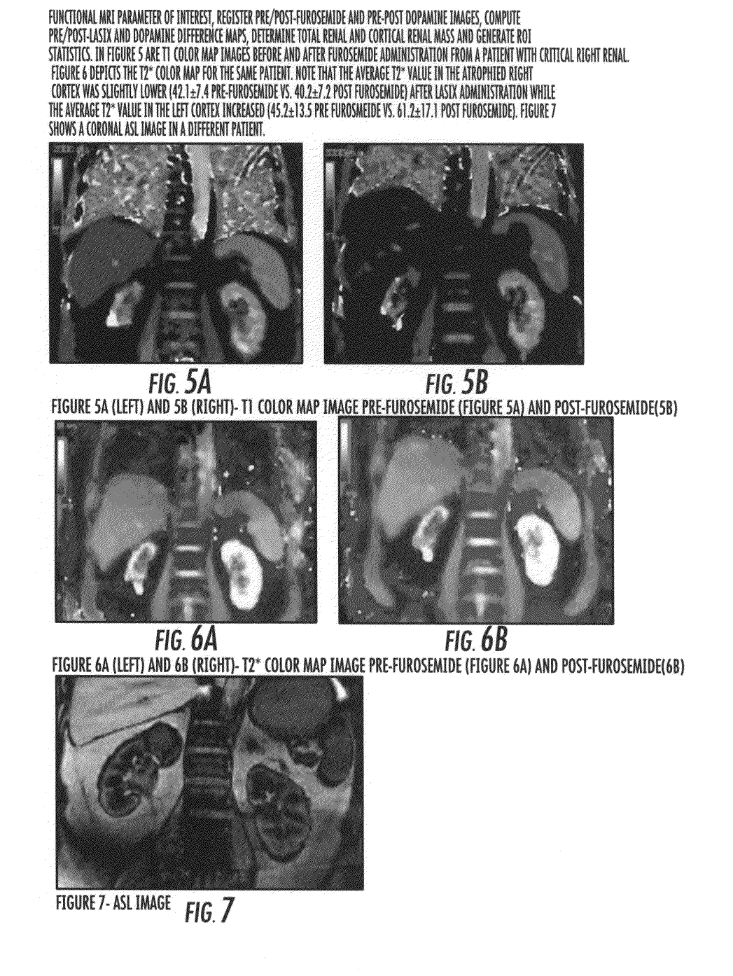

FIGS. 5A and 5B are T1 color maps (shown in grey scale) with the pre-agent (furosemide) T1 color map shown in FIG. 5A and the post-agent (furosemide) T1 map shown in FIG. 5B according to embodiments of the present invention. Functional MRI parameters can be evaluated using pre/post furosemide and pre/post dopamine images, difference maps of each pre/post image set can be computed. Total renal and cortical renal mass can be electronically calculated. The T1 analysis can be configured to determine if renal fibrosis is present. FIGS. 5A and 5B are maps of a patient having critical right renal artery stenosis.

FIGS. 6A and 6B are T2* color maps of the same patient shown in FIGS. 5A and 5B (shown in grey scale) with the pre-agent map shown in FIG. 6A and the post-agent map shown in FIG. 6B (using the same agent used to generate FIG. 5B) according to embodiments of the present invention. The average T2* value in the atrophied right cortex was slightly lower after furosemide while the average T2* value in the left cortex increased 45.2+/−13.5 to 61.2+/−17.1.

FIG. 7 is a coronal ASL image of a different patient, which illustrates the differences in the image types.

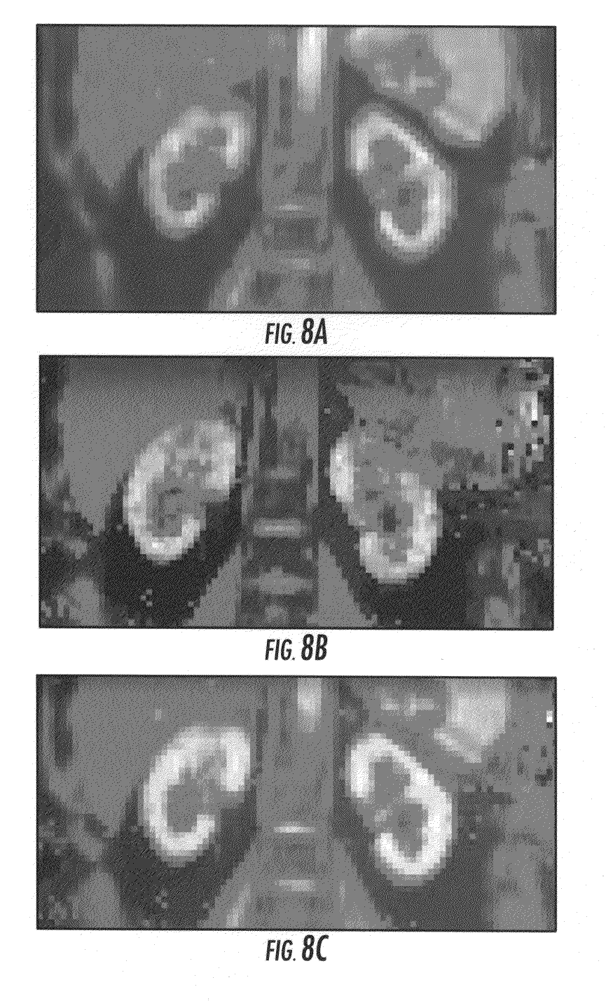

FIGS. 8A-8C are exemplary color coded tissue maps that can be simultaneously or selectively shown on a display associated with a workstation according to embodiments of the present invention. FIG. 8A is a T1 map. FIG. 8B is a T2 map. FIG. 8C is a weighted-sum map of the maps of T1 and T2 (of the corresponding pixels/voxels) of according to embodiments of the present invention. The weighted sum image W (bottom image=W) of a T1 map (top image=T1) and a T2 map (middle image=T2) can be expressed by Equation (1):

W=w1*T1+w2*T2, Equation (1)

where w1=1 and w2=1, in this example.

However, other weights can be used and the weights can be less than 1 and greater than 100, e.g., typically a scalar value from about 0.1-10. It is noted that w1 can be larger than w2 or w2 can be larger than w1. Each weight can be the same or different and greater or less than 1.

One or more tissue maps can be selectively altered by allowing a user to apply different weights. Different weights may automatically be applied or a user may select one from a define range or pull down menu of options or other UI options.

A pixel by pixel ratio can be computed for the maps producing a ratio map of pre- and post-rug or agent administration. The average T1 and/or T2* can be computed for the cortex and medulla in both a pre-drug or pre-agent map and a post-drug or post-agent map. The ratio can be computed producing a scalar average T1 and/or T2* ratio for the cortex and the medulla.

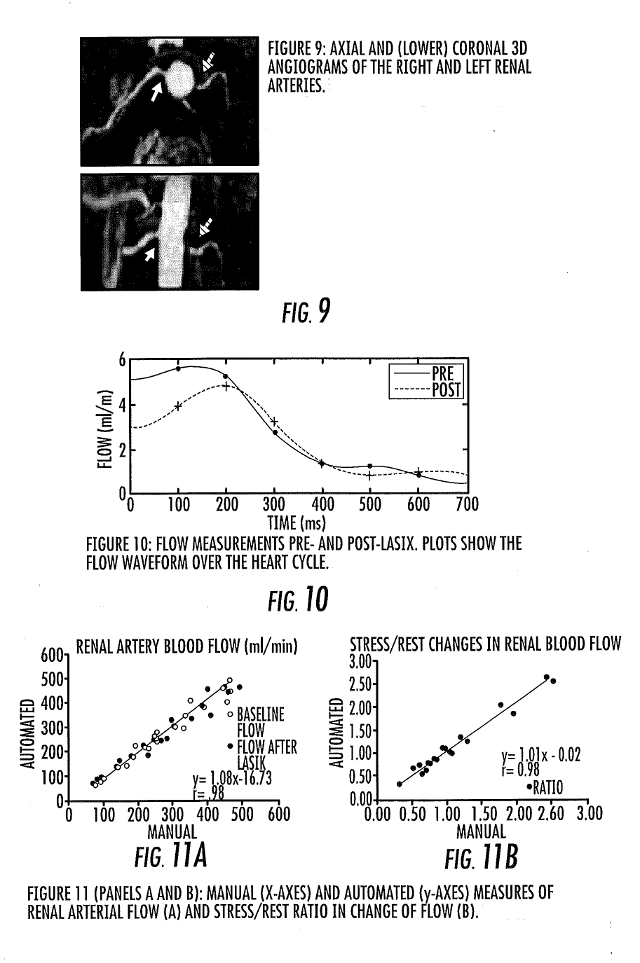

FIG. 9 are axial and lower coronal 3D angiograms of right and left renal arteries with visual indicia (e.g., arrows) showing near total occlusion of the left artery and about 50% stenosis of the right artery (the more severe occlusion can be shown in a different color or opacity for visual emphasis) according to embodiments of the present invention.

FIG. 10 is a graph of flow (ml/min) versus time (ms) of flow measurements over a cardiac cycle illustrating pre- and post-agent (LASIX) administration flow rates according to embodiments of the present invention. Mean flow increased from 132 to 149 ml/min.

FIGS. 11A and 11B are graphs of manual versus automated renal artery blood flow (ml/min) and stress/rest changes in flow (FIG. 11B) according to embodiments of the present invention (in use, the automated analysis may be shown without the manual flow calculation, as the manual one is shown for comparison as to accuracy). Each dot represents a measurement point from one individual in the study. As shown, there was very high correlation between flow and flow ratio changes among the participants in the study.

FIG. 12 is a renal image showing four different parameters of the kidney that can be shown simultaneously or concurrently on a display for ease of diagnosis according to embodiments of the present invention. These include: (1) blood flow supply which can be measured with phase contrast MRI; (2) renal artery patency, which can be measured with 3D MRI angiogram (see, e.g., U.S. Pat. No. 7,283,862 for a description of Rapid Multi-Slice Perfusion Imaging, which may be suitable for renal perfusion and/or angiographic analysis, the contents of which are hereby incorporated by reference as if recited in full herein); (3) intra-renal vascular oxygenation, which can be measured with multi-echo T2* MRI; and (4) intra-renal tissue oxygenation, which can be measured with multi-echo T1 MRI. Change in flow before and after oxygenation can be evaluated and provided as additional data on reserve capacity. These results can be provided rapidly for immediate evaluation, post-scan, e.g., in under 1 hour, typically in about 5-45 minutes.

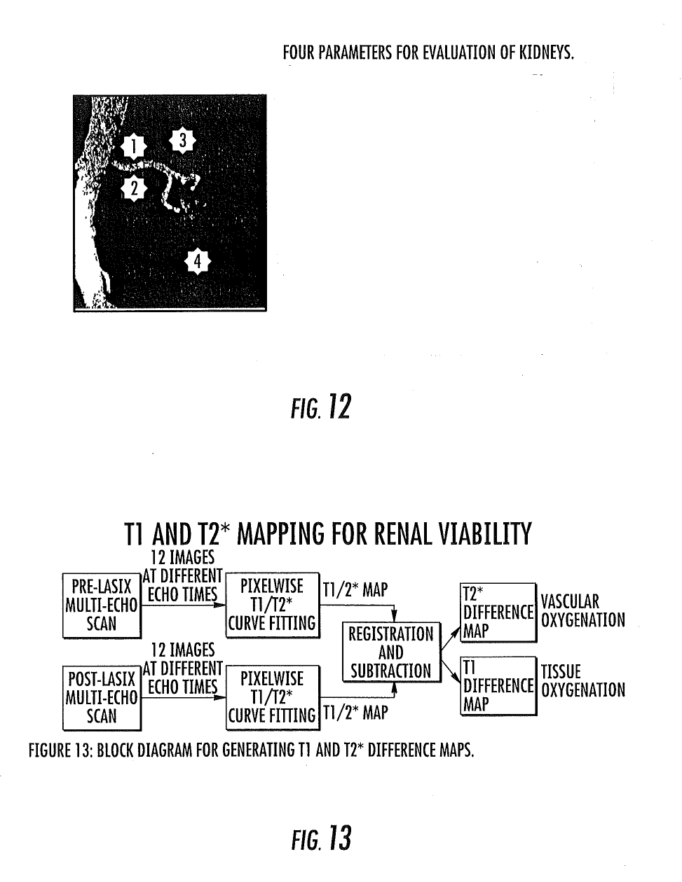

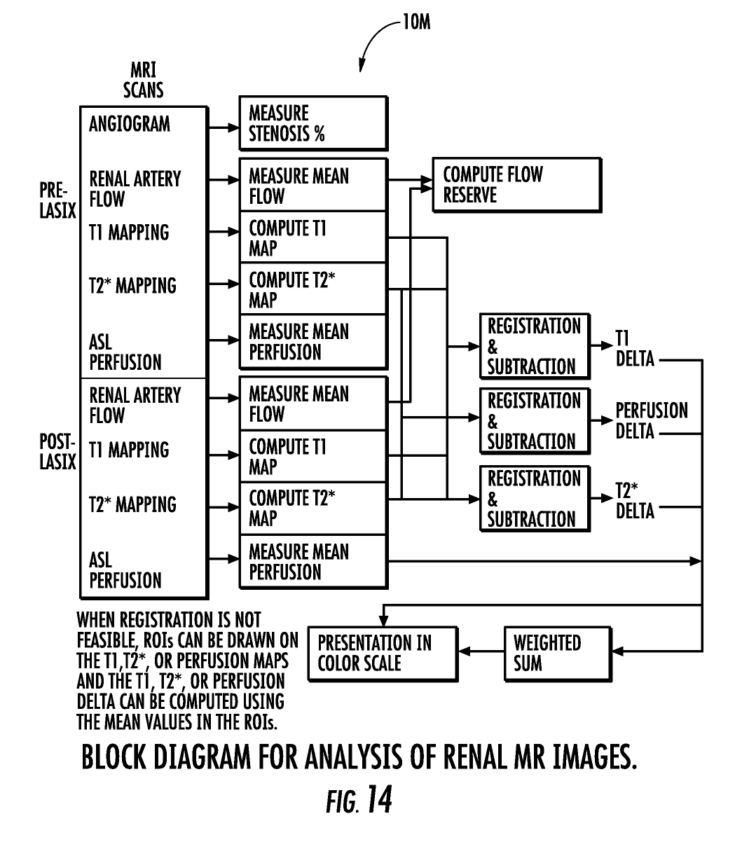

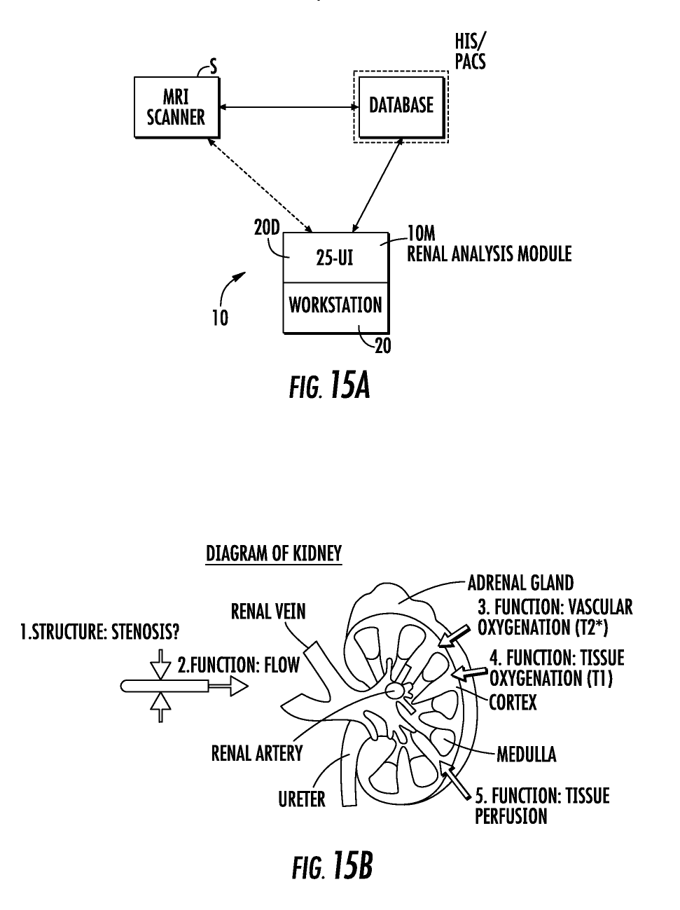

FIG. 13 is a block diagram/flow chart of exemplary renal (T1 and T2* difference maps) tissue mapping using T1 and T2* MRI image data for renal viability assessment according to embodiments of the present invention. Pre and post Lasix multi-echo scans (such as 12 images at different echo times) can be obtained. The T1 and T2* data can be pixel wise curve fitted in a similar manner to generate respective T1 and T2* maps. The maps can be registered to yield a difference map for T1 indicating change in tissue oxygenation, and T2* representing change in vascular oxygenation. For maps with poor registration, ROI analysis can be used to compute the T1 and T2* regions in the kidney pre and post LASIX. Each of these difference maps can be provided to a clinician on a display.

FIG. 14 is a block diagram of an automated analysis circuit for renal evaluation using MRI data according to embodiments of the present invention. Similar to the T1 and T2* maps shown in FIG. 13, a perfusion difference map may also be generated. The renal evaluation circuit or module 10M/360 can be configured to provide a measure of stenosis, a measure of mean perfusion and generate a weighted sum tissue map that combines the difference maps to generate a composite map in color scale reflecting the measures of oxygenation and perfusion from each of the difference maps, e.g., the tissue and vascular oxygenation and the perfusion difference maps.

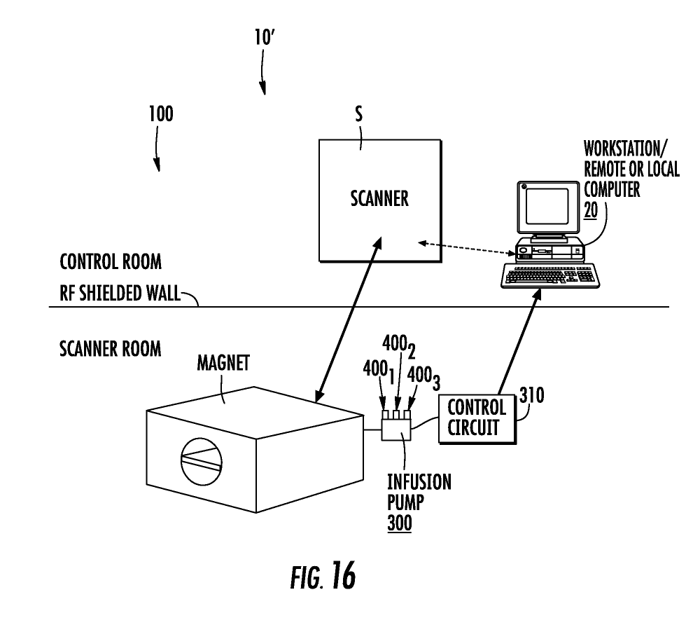

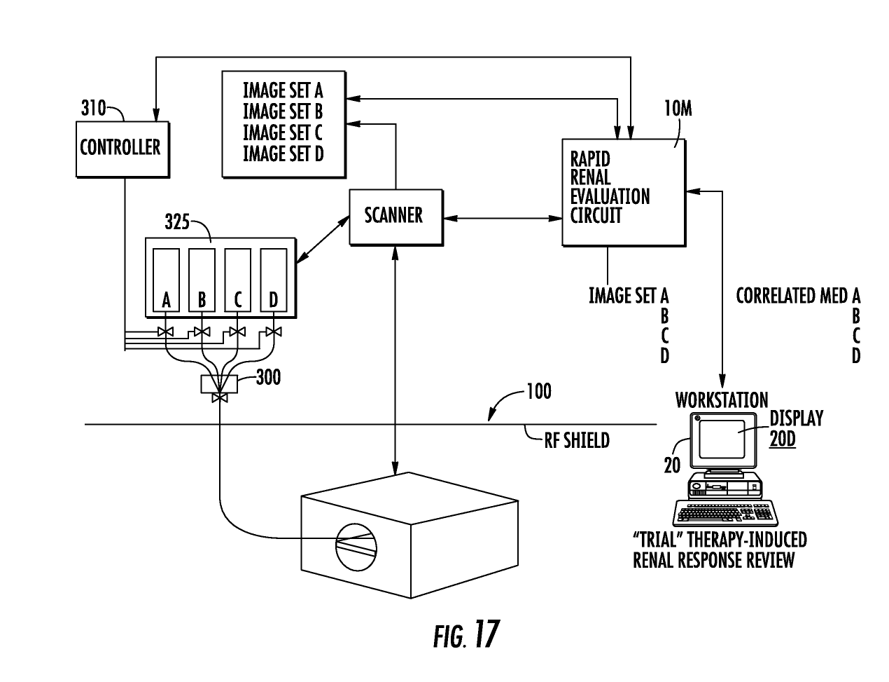

FIG. 15A is a schematic illustration of a renal evaluation system that uses MRI data according to embodiments of the present invention.

FIG. 15B is an exemplary prophetic section view of a kidney that shows different tissue parameters obtained using MRI data according to embodiments of the present invention.

A first image of a region of interest of tissue of a patient can be obtained. An image may be obtained, for example, by acquisition of the image from an imaging system, such as the imaging systems discussed above, and/or by obtaining the image from a database, file or other storage of the image data. For example, a patient's images may be maintained in a historical database, e.g., patient records database such as PACS and/or HIS, for subsequent recall. The region of interest of tissue in a patient that is imaged may, for example, kidney or portions thereof. In particular embodiments of the present invention, the tissue may be human tissue. In other embodiments, the tissue may be animal tissue.

A second image of the tissue in the region of interest can be obtained. The second image may be acquired and registered (taken at the same slice locations) with the corresponding first image. The second image may also be obtained as described above with reference to the first image. Thus, for example, images may be historical images as well as recently acquired images.

The first image and the second images can be evaluated to determine one or more renal tissue characteristic of the images. The characteristic of the images may, for example, be an average intensity of pixels/voxels in the region of interest. The characteristic of the pixels/voxels that is evaluated may include intensity, color, saturation and/or other characteristics of individual pixels/voxels as well as relative characteristics of multiple pixels/voxels, such as ratios, differences of pixel or voxel values between two or more images, and the like.

The results of this evaluation can be automatically, electronically generated and may be provided to a user in a report format electronically on a display or in other suitable (e.g., print form) or may be provided for further analysis. The results can be pattern matched to a library of patterns that are characteristic of particular kidney injuries, diseases and/or conditions or that can predict positive or negative outcomes of one or more defined therapy alternatives, such as whether the patient is a good candidate for surgical intervention or a particular drug therapy.

The results of the determination may, for example, be provided as part of a graphic user interface to a display associated with the workstation.

In still further embodiments of the present invention, the evaluation of image data, i.e., the intensity or other characteristic of the pixels of different kidney images, may be performed automatically or partially automatically utilizing image processing techniques. An automatic comparison may, for example, also include registration of the differing images to each other. Such a registration may be provided utilizing conventional pattern recognition and/or alignment techniques such that corresponding pixels of the images or portions of the images are each associated with approximately the same physical location within the patient.

In particular embodiments of the present invention, a patient may be taken to the MRI suite where he/she will typically be placed supine on the MRI table. MRI scans may be performed on, for example, a 1.5 or 2.0 T Tesla GE or Siemens scanner or another MRI scanner.

Upon, after and/or during image acquisition during a patient MR Scanner session, the image data may be transferred electronically to a renal analysis circuit, module or database. This information may be available to the MRI technologist or clinician via a workstation such as at a display associated with a workstation with a computer or processor at the time of each scan or subsequent to some or all acquisitions. In some embodiments, the user can indicate a region for use in registration of serial images to facilitate the location or adjustment of slice positions (registration).

Whether a parameter or tissue characteristic is shown or identified in a respective renal tissue map as being impaired, degraded or otherwise abnormal or affected by a therapy versus normal or untreated conditions can be based on the relative or absolute measure of the respective pixel or voxel, not limited to intensity of pixels, of the tissue characteristic of the patient itself, based on a baseline tissue map or MRI images, or comparison of different MRI images taken at different times or in response to different therapies or challenges, or based on predefined values or ranges of values associated with a population “norm” of typical normal and/or abnormal values relative to gender, age and the like, or combinations of the above.

In some embodiments, the UI 25 can be configured to allow a clinician to increase or decrease the intensity or change a color of certain tissue characterization types, e.g., to show a region of interest with a different viewing parameter, e.g., in high-contrast color and/or intensity, darker opacity or to fade certain image features from view and the like. The tissue map can comprise MR image data that reflects a change in a tissue property obtained after or during the MR scan session procedure, e.g., using an administered challenge such as LASIX, or other therapeutic agent or other therapy and the like.

Multiple interventional factors can be assessed substantially simultaneously during the image acquisition and/or rendering process. In some embodiments, more than one agent can be administered, e.g., lasix and a concomitant medication like Dopamine or Dobutamine that improves renal blood flow. The combination of these agents may be more effective at selecting kidneys that will improve function after successful interventions.

The diuretic selected for a particular patient may vary depending upon the segment of the kidney (cortex versus medulla) that is being assessed. Agents such as hydrochlorothiazide, another diuretic, may be more efficacious than lasix in some individuals as this agent preferentially assesses the cortex.

The analysis operations can be carried out electronically to generate an evaluation summary or report of kidney status. The report can be an electronic and/or paper report, and may be generated in substantially real-time or shortly after acquisition of the image data.

Some embodiments of the invention may be used to evaluate how drugs affect kidney function and/or tissue for pharmacological studies, such as, for example, clinical trials and/or drug discovery.

FIG. 15A illustrates an exemplary image processing system with a renal analysis module or circuit 10M.

FIG. 15A illustrates that the system 10 can include at least one workstation 60 that has a portal for accessing the module 10M or that is onboard or partially onboard the workstation. The module 10M can be held on a local server or at least one processor or a remote server or at least one processor accessible via a LAN, WAN or Internet. The workstation 20 can communicate with archived patient image data which may be held in a remote or local server or other electronically accessible database or repository. The workstation 20 can include a display with a GUI (graphic user input) 25 and the access portal. The system 10 can communicate with or be integrated into a PACS system. The workstation 20 can allow interactive collaboration of image rendering to give the physician alternate image views of the desired features. The map rendering circuit, module or system can be configured with the GUI or other UI to allow a user to zoom, rotate, and otherwise translate to give the physician visualization of the patient data in one or more views, such as section, front, back, top, bottom, and perspective views.

The map rendering system may be wholly or partially incorporated into the physician workstation 20, or can be a remote or local module (or a combination remote and local module) component or circuit that can communicate with a plurality of physician workstations (not shown). The visualization system 10 can employ a computer network and may be particularly suitable for clinical data exchange/transmission over an intranet. The workstation can access the data sets via a relatively broadband high speed connection using, for example, a LAN or may be remote and/or may have lesser bandwidth and/or speed, and for example, may access the data sets via a WAN and/or the Internet. Firewalls may be provided as appropriate for security.

The module 10M can be at least partially integrated into the control cabinet associated with an MR Scanner with image processing circuitry. Although not shown, part of the module 10M can be held in both the Scanner S and one or more workstations 20, or totally on one or more remote circuits or totally in a workstation 20, which can be remote or local.

FIG. 16 illustrates an example of a conventional MRI suite 100 that includes a control room with MRI Scanner operating components such as an RF amplifier and control circuits in one or more cabinets, the MRI Scanner “S”, and a separate adjacent room or chamber holding a high field magnet in which a patient is placed for an MRI procedure (typically called the Scanner room). An RF-shielded wall and/or penetration panel separates the two rooms. RF Shielding is important because it isolates the MRI scanner from external RF sources that can cause artifacts in the MRI image. For a typical MRI scanner chamber or room, the RF shielding causes at least 100 dB of signal attenuation of signals in the frequency range of 1 Hz to 150 MHz. Holes or openings made in this shielding can compromise the shielding effectiveness.

As is also known, in order to allow access in the MRI scanner chamber for non-metallic conduits of water, medical gas or optical data lines, special waveguides can be installed in the RF shielded room. On the outside, these waveguides are typically electrically connected to the room shielding. Waveguide depth and diameter is based on the fact that an electromagnetic field attenuates rapidly down a small diameter hole of sufficient depth, providing certain conditions are met. Using the waveguide in this manner is commonly called ‘waveguide below cutoff’. This guide allows small diameter holes to be made in conductive enclosures, as may be needed for ventilation, or as a pass-through for non-metallic members. In addition, RF filters are typically mounted on the RF shield and create a penetration point for electrical power, data cables and the like. This is typically carried out using a removable portion of the RF shield which is called a penetration panel.

The system 10 can be configured to generate a relatively rapid analysis of renal response due to one or more test (sub-bolus amount) or therapeutic amount/bolus dose of a therapeutic agent.

Referring again to FIG. 16, in some embodiments, the renal evaluation system 10′ can include an infusion pump 300 in communication with at least one test dose of a therapeutic agent 400 (shown as three different agents 400₁, 400₂, 400₃, but more or less therapeutic agents 400 can be used). Examples of MRI-compatible infusion pumps are described in one or more of U.S. Pat. Nos. 5,494,036; 7,221,159; 7,283,860; and U.S. Patent Application Publication No. 2008/0015505, the contents of which are hereby incorporated by reference as if recited in full herein.

The term “test dose” refers to a sample and/or sub-bolus amount of a therapeutic agent. The test dose can have a short half life, at least in the kidney; e.g., it is typically substantially gone from the kidneys in between about 5-10 minutes from cessation of the delivery of the respective agent, at least in an amount that causes or induces any significant renal response. The test dose may be in an alternate formulation from day to day or prescribed conventional usage, e.g., which is typically by way of oral administration such as pills or tablets. The test doses are typically substantially pharmaceutically equivalent formulations of conventional therapeutic agents, formulated for IV administration.

The test dose can be provided in any suitable amount, typically in an amount sufficient to allow for between about a 1-10 minute IV administration to a subject (e.g., typically a human patient) using, for example, an infusion pump. Two or more the test doses may be serially administered in a relatively rapid manner, e.g., in under about 1 hour, and MRI image data obtained based to evaluate a patient's renal function/response.

Some of the test doses may be administered concurrently for combination evaluation while others may be administered alone.

In some embodiments, all the test doses are delivered individually, with or without a diuretic or other stress/challenge agent.

In some embodiments, each agent can be successively administered with a short transition time between each agent, such as between about 10 seconds to about 15 minutes, more typically between about 1 minute to about 5 minutes, between successive test doses. Saline or other “wash” liquid may be administered between each serial administration.

The therapeutic agents 400 may be for treating renal conditions or may be for treating other conditions that might have an impact on renal function, at least in some patients. In some embodiments, a combinatorial agent treatment may be contemplated and evaluating renal response to a planned combination may be beneficial. The renal evaluations may also have benefit in drug discovery and/or clinical trials.

For example, some patients presenting with diabetes, high blood pressure, heart disease, asthma, COPD, infections, or other conditions may have a number of therapeutic treatment options available; however, some of these may present a risk of renal injury or dysfunction, or otherwise negatively impact renal function. Providing test-dose MRI-based renal response screening of different drug options can allow a clinician to make more informed treatment decisions for a particular patient thereby inhibiting renal injury induced by a treatment.

The system 10 includes a control circuit 310 in communication with the infusion pump 300 to allow for active “on”/“off” serial delivery of respective therapy agents 400. The control circuit 310, and indeed the pump 300, can reside in the Scanner room or in the control room (FIG. 17). The infusion pump 300 can include remote or onboard valves, manifolds, sensors and the like that allow the automated and selectively controllable serial delivery of the different test doses. The circuit 310 can include an automated module to (i) communicate with the MR Scanner to synchronize MRI Scanner pulse sequences and/or signal acquisition to a drug administration; and/or communicate (ii) with the renal evaluation circuit or module to correlate what MRI images correspond to a particular agent for rapid analysis. The analysis of one image set related to one drug can be carried out electronically while image signal of another images set related to a second drug is being obtained.

FIG. 17 illustrates that the infusion pump 300 can include or be in communication with a housing 325 that encloses a plurality of test doses 400₃, 400₂, 400₃, 400₄. The housing can communicate with the Scanner and/or controller (control circuit) 310 so that Image sets A, B, C, D, can be correlated to a particular agent A, B, C, D (or combination of agents). The renal evaluation circuit 10M can be in communication with a workstation 20 having a display 20D to provide a display or other report output of “trial therapy-induced” renal responses. The test doses 400 and pump 300 are shown in the control room, but can be, and typically are, in the Scanner room.

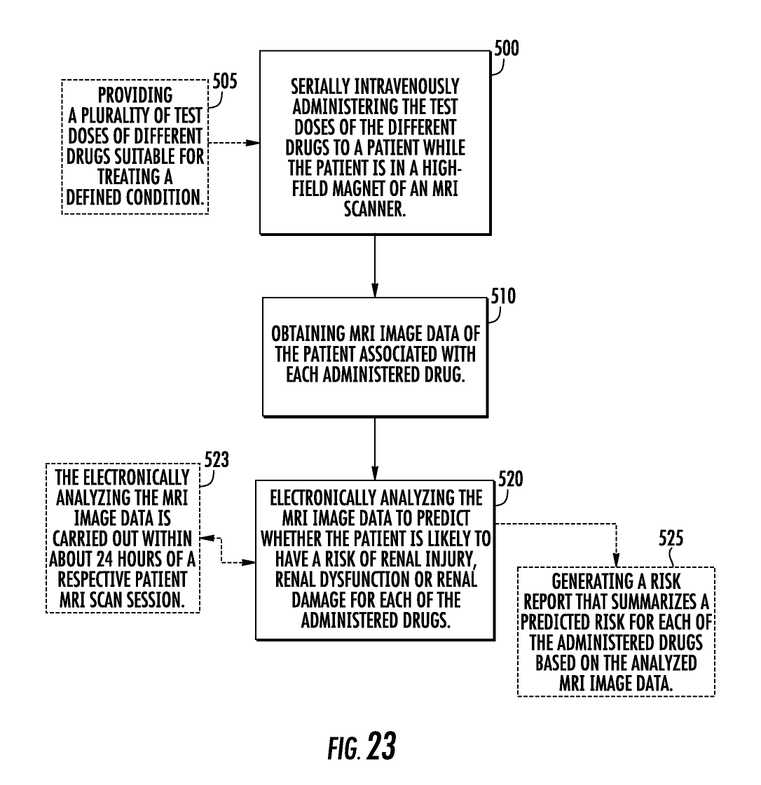

The renal evaluation circuit 10M can include or be in communication with an electronic library module 10L (FIG. 22). The electronic library module 10L can include a list defined conditions and a list of different therapeutic agents correlated to the different defined conditions. A user can select a condition from the defined conditions and the circuit 10M can present associated different therapeutic agent options for consideration to the display. The library of different conditions 10L can include at least two of the following conditions: diabetes, COPD, asthma, heart failure, heart disease, chemotherapy, infection, and high blood pressure.

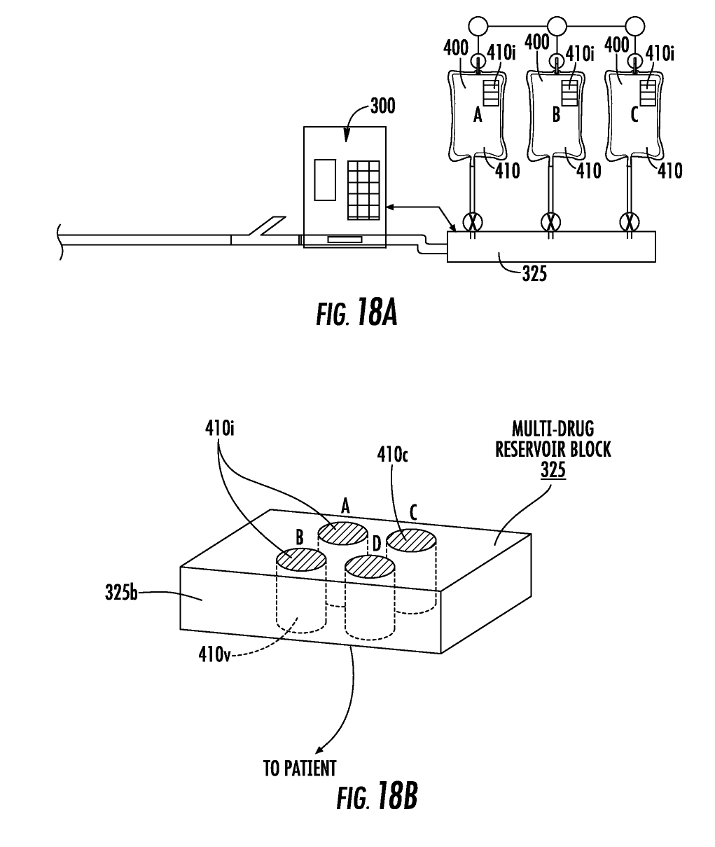

FIG. 18A illustrates that the system 10 can include a holding member 325 such as a housing that can receive a plurality of different agents in different channels or spaces and controllably deliver one or combinations. The correlation as to what agent is in what location and/or as to what agent is delivered with respect to a set of MRI image slices can be made by having a person enter the data or use an optical reader that scans a barcode, such as a QR (quick response “matrix” barcode) or other barcode associated with the test dose package or label.

In some embodiments, the control circuit 310 and/or the holding member 325 can include onboard readers and sensors that provide the desired identification data and time of delivery for correlation of obtained image data. That is, each test dose may have electronically readable indicia that allow an electronic reader to identify the agent and correlate the agent to a position in the body of the holder 325. The indicia 410i can be a barcode on the cap 410c of a vial 410v (FIG. 18B) or on a surface of a pouch 410p (FIG. 18A) or other tag, label or location of the test dose.

FIG. 17 illustrates that the housing is configured to hold the pouches 410p in an enclosure which can be locked after loading to inhibit tampering and the like. FIG. 18A illustrates the pouches 410p may be suspended and directed to release their contents into a manifold for delivery to a patient or other subject. FIG. 18B illustrates that the holder 325 can be a block 325b that receives vials 410v of the agents 400. The holder 325 can include onboard flow paths, valves and the like and/or may connect to conduits for fluid delivery.

The circuit/module 10M and/or module 350 (FIG. 3) can evaluate a baseline set of MRI image slices with respect to each test dose evaluation to determine a change in one or more renal functions from that baseline or a “stress challenge” state (e.g., using a difference map) such as using a difference map of T1, T2, T2* and/or a difference map of ratios of one or more of these parameters.

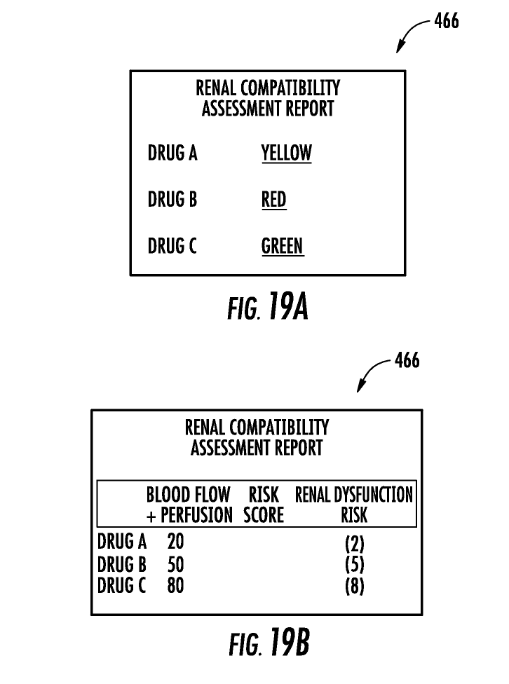

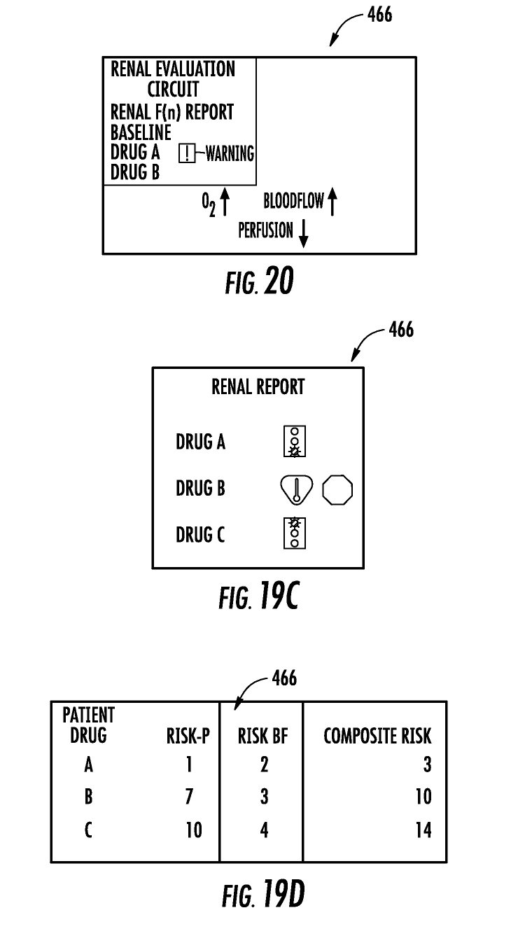

FIGS. 19A-19D illustrate exemplary renal evaluation reports 466 of different test agents. The reports 466 can be transmitted electronically for display and/or by paper. FIG. 19A illustrates that each test dose of agent evaluated can be “graded” with a color that identifies potential risk of kidney complications, injury or dysfunction for that agent, e.g., “green” for no undue risk identified (or potentially even a positive impact on renal function), yellow for an indication of some or moderate risk and “red” for an increased or high risk. Thus, the risk report 466 can include a color risk evaluation for each of the different therapeutic agents ranging from high to low risk of kidney complications or undesired kidney response, including “green” for low risk, “yellow” for a moderate risk, and “red” for a high risk.

FIG. 19B illustrates a numerical risk score can be used to provide renal function responses, e.g., a relative risk score rating between 1-100 as shown with an optional alternative 1-10 scale (shown in parenthesis) and the like. This score can reflect the agent's impact on blood flow and perfusion and optionally oxygenation as well. In some embodiments, different risk scores can be used for each of perfusion, oxygenation and blood flow. A high score can reflect a higher risk. However, the risk scale can be configured in the reverse with a high score indicating a low risk.

FIG. 19C illustrates that the report 466 can include visual icons that indicate risk, such as a “stoplight” or warning sign where appropriate for different drugs.

FIG. 19D illustrates that the report 466 can include risk scores for each of several categories including renal artery blood flow (BF), perfusion and a composite score. The composite risk score can be an un-weighted sum of individual risk scores (as shown) or a weighted sum.

The reports 466 can also be provided using combinations of risk scores and color risk indicia.

FIG. 20 is an example of a report 466 that can be generated for test doses of agents selected to treat renal injury or dysfunction. The report can include a baseline evaluation and/or the test dose evaluations can be determined based on change in one or more renal functions from that baseline, such as using a difference map of T1, T2, T2* or ratios of one or more of these parameters. The increase in different measures of renal function (oxygenation, perfusion and renal artery blood flow) can be provided to allow a user visual feedback on the response. Measures can consider both medulla and cortex regions. The increase/decrease from baseline or between different agents can be scaled and provided in a graphic output.

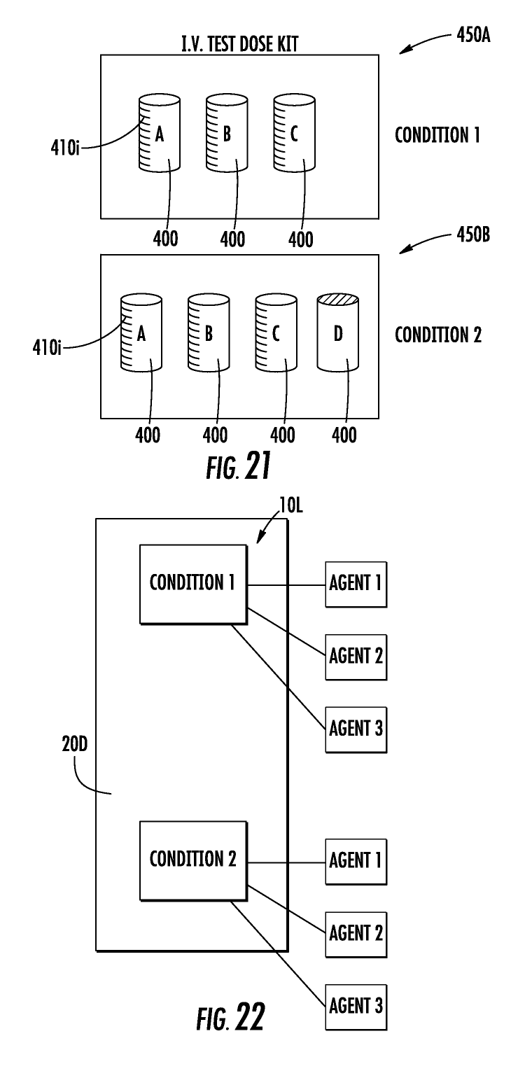

FIG. 21 illustrates that test doses 400 can be provided in kits 450A, 450B of test vials or pouches populated depending on the condition being treated and/or the patient, shown as Condition 1 and Condition 2. Condition 1 can be, for example, diabetes, and Condition 2 can be, for example, high blood pressure. A set of different agents may be packaged together in a kit 450 and a clinician may select a subset of those agents for test dose evaluation for any particular patient. Thus, a plurality of different drugs for a noted condition (e.g., diabetes) can be evaluated for their respective effect on renal function so as to allow a clinician to select a drug for treating the condition balanced with its effect on renal function so as to avoid drugs with unfavorable or negative effects (or one with the least negative effect).

FIG. 22 schematically illustrates different therapeutic agents or drugs (agents 1, 2, 3) can be evaluated for a particular condition. A display 20D may provide an electronic library module 10L of defined conditions and a list of different therapeutic agents correlated to the different defined conditions. A user can select a condition from the defined conditions and the circuit 10M can electronically and/or programmatically present associated different therapeutic agent options for consideration to the display. The library of different conditions can include at least two of the following conditions: diabetes, COPD, asthma, heart failure, heart disease, chemotherapy, infection, and high blood pressure.

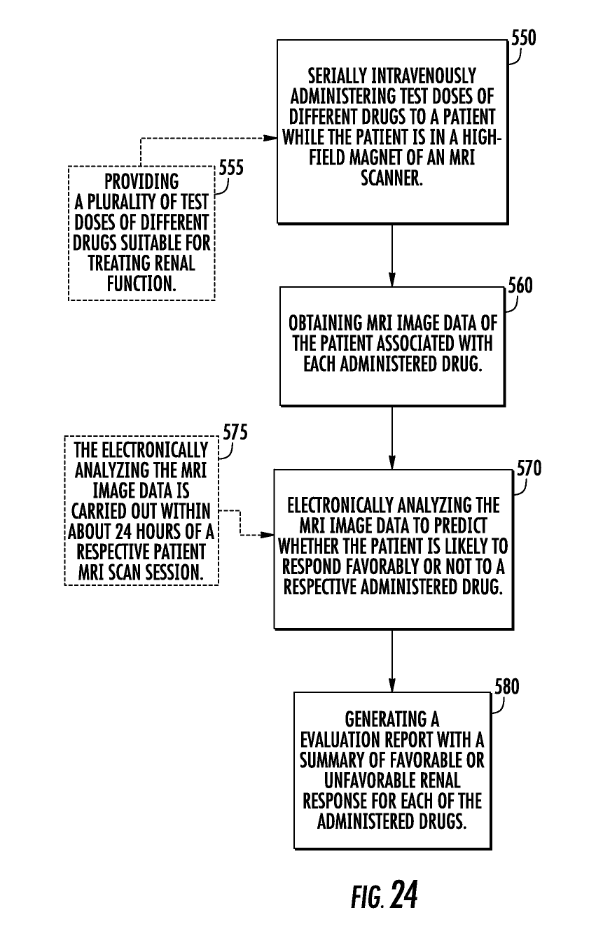

Referring now to FIG. 23, some embodiments are directed to methods of screening patients to inhibit potential renal complications associated with a drug therapy. The methods can include: serially intravenously administering test doses of different drugs to a patient while the patient is in a high-field magnet of an MRI Scanner (block 500); obtaining MRI image data of the patient associated with each administered drug (block 510); and electronically analyzing the MRI image data to predict whether the patient is likely to have a risk of renal injury, renal dysfunction or renal damage for each of the administered drugs (block 520).

The method can also include generating a risk report that summarizes a predicted risk for each of the administered drugs based on the analyzed MRI image data (block 525). Optionally, the method can include providing a plurality of test doses of different drugs suitable for treating a defined condition (block 505). The electronically analyzing the MRI image data can be carried out within about 24 hours of a respective patient MRI scan session (block 523).

The defined condition can be one of diabetes, COPD, asthma, heart failure, heart disease, chemotherapy, infection, and high blood pressure.

The electronically analyzing can determine a measure of blood flow in a renal artery, a pattern of oxygenation and a pattern of perfusion for each of the administered agents. Composite maps showing, for example T1, T2, T2* and perfusion may be generated and displayed.

Referring now to FIG. 24, methods of selecting a drug therapy for improving renal function can include: serially intravenously administering test doses of different drugs to a patient while the patient is in a high-field magnet of an MRI Scanner (block 550); obtaining MRI image data of the patient associated with each administered drug (block 560); electronically analyzing the MRI image data to predict whether the patient is likely to respond favorably or not to a respective administered drug (block 570); and electronically generating an evaluation report with a summary of favorable or unfavorable renal response for each of the administered drugs (block 580). Typically, the analyzing and generating are carried out in a rapid fashion (block 575). The term “rapid” refers to evaluations and reports that are generated within about 24 hours and more typically within about 2 hours, such as between about 30 minutes to about 2 hours after a respective subject or patient MRI scan session. A plurality of test doses can be provided for the serially administering step (block 555).

In some embodiments, the automated system 10 evaluate the interrelationships of the acquired parameters including renal oxygenation (e.g., through a measurement of T2*) and renal function using the arterial spin labeling technique. Embodiments of the invention can automatically (electronically and/or programmatically) identify the regions, cortex or medulla, as well as the relationship of oxygenation to perfusion within the regions.

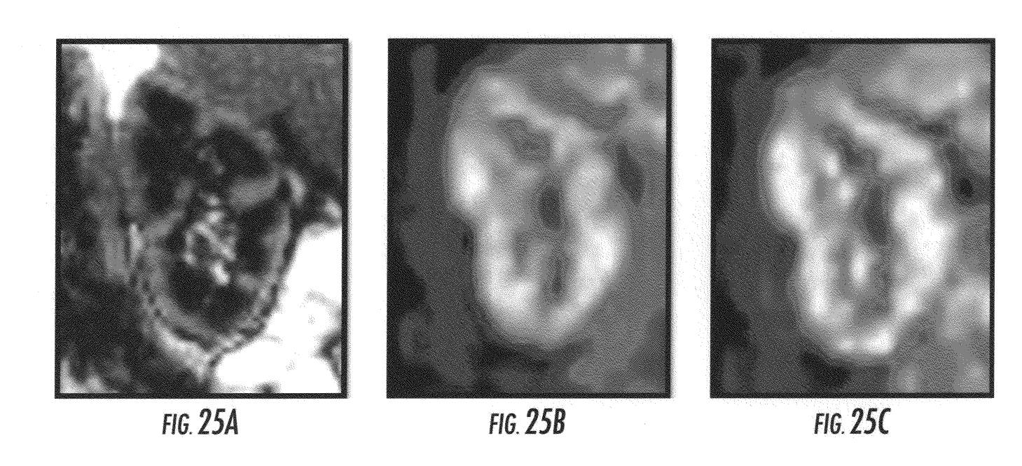

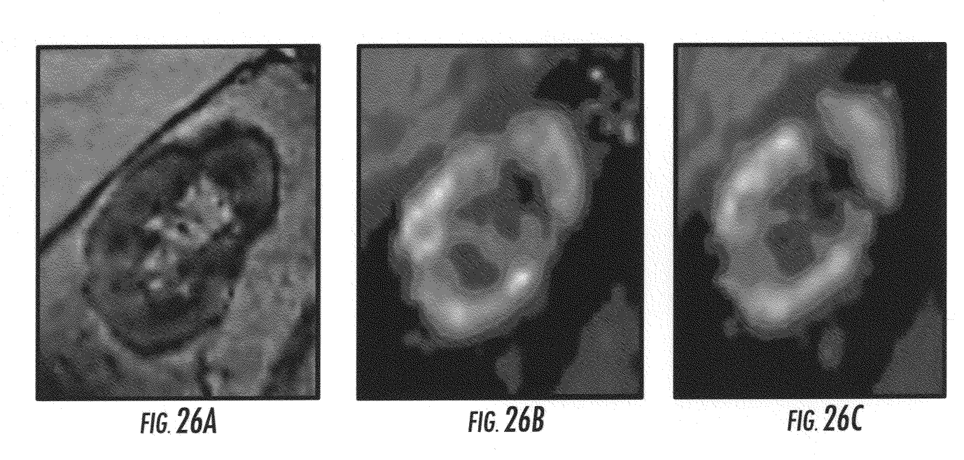

FIGS. 25A-C illustrate one patient (77 year old Caucasian female with hypertension and sever bilateral RAS) with a “normal response” and increase in post T2* image intensity while FIGS. 26A-C illustrate a different patient (75 year old Caucasian male with hypertension, diabetes and chronic renal insufficiency with an estimated GFR of 37 ml/min/1.73 m²) with an abnormal response and decrease in post T2* image intensity. FIGS. 25A (right kidney) and 26A (left kidney) are arterial spin labeling images of the respective kidney. For the patient images in FIGS. 25A-C, cortical T2* values increased from a mean of 65.2 ms to 71.5 ms and medullary T2* values increased from 53.5 ms to 59.5 ms in response to furosemide administration (compare pre in 25B to post in 25C). For the patient images in FIGS. 26A-C, cortical T2* values decreased from a mean of 55.4 ms to 52 ms and medullary T2* values decreased from 40.3 ms to 38.2 ms in response to furosemide administration (compare pre in 26B to post in 26C).

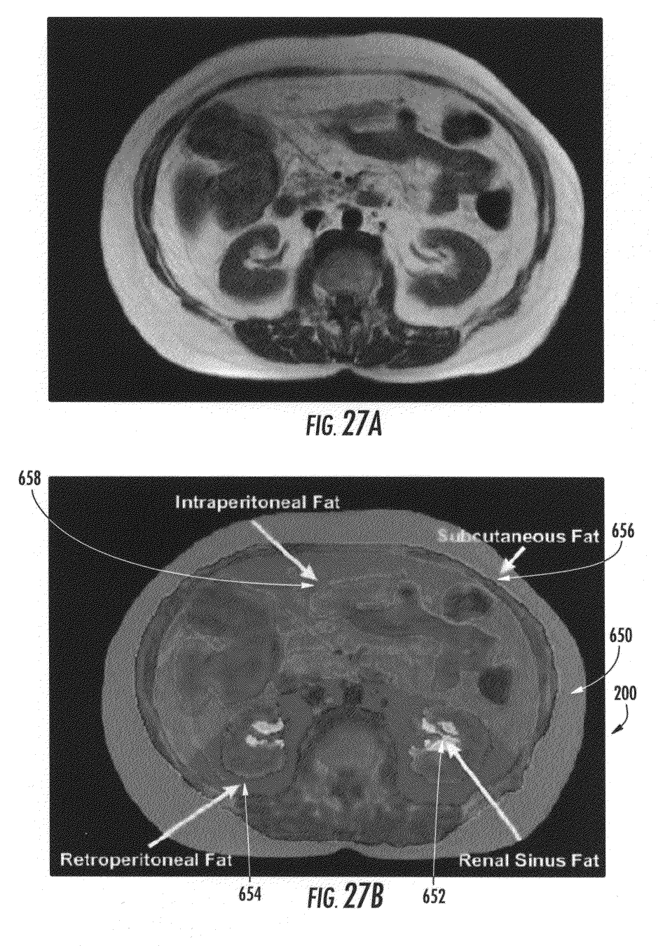

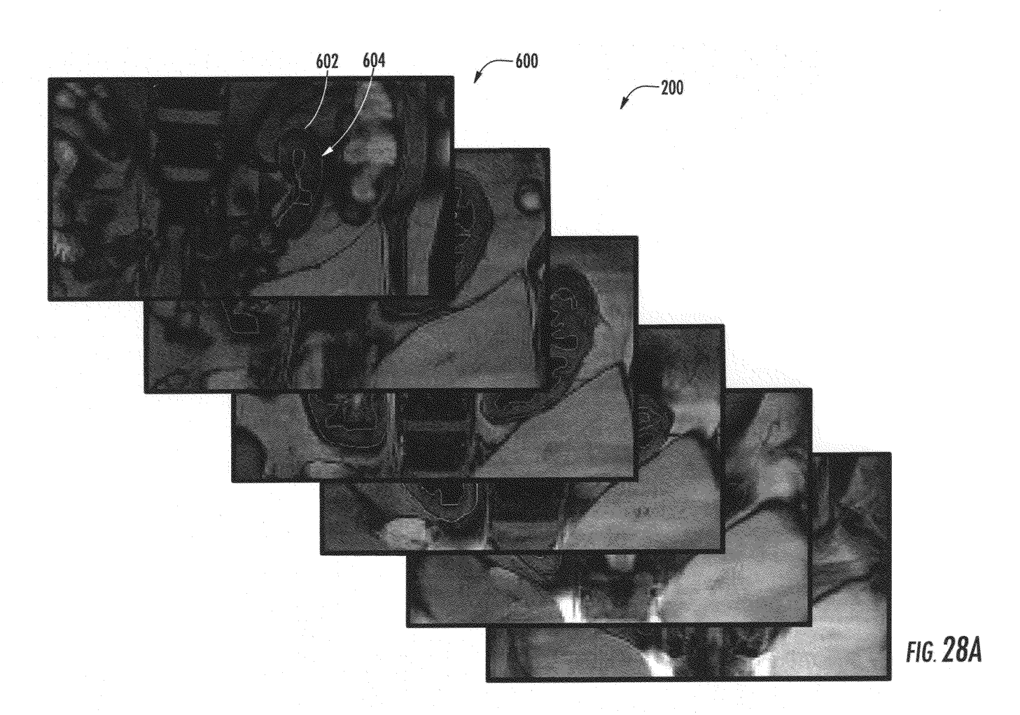

In some embodiments, the automated system can evaluate structures that surround the kidney, particularly different volumes of fat such as shown in FIG. 27B. Adverse structures include the accumulation of perirenal fat. The accumulation of this perirenal fat within the hilum of the kidney appears to restrict blood flow in the low pressure conduits (ureter, and systemic vein) and therefore may promote high intra-renal pressures and further renal damage. FIG. 27A illustrates an axial image acquired from a participant at the second lumbar vertebral body. FIG. 27B illustrates the same image color coded to tissue type to visually emphasize the different fat volumes or segments that can be shown on a display 20D. The color coded image 650 can show, for example, renal sinus (RS) fat 652, retroperitoneal (RP) fat 654, subcutaneous (SC) fat 656 and intraperitoneal (IP) fat 658. The color coded image 650 may also show the viscera, musculature and vertebra bodies in one color (e.g., red) while the different fat volumes or regions are shown in different colors (or different shades of color or even with other visual indicia such as hash marks, or other visual contrast or mapping techniques).

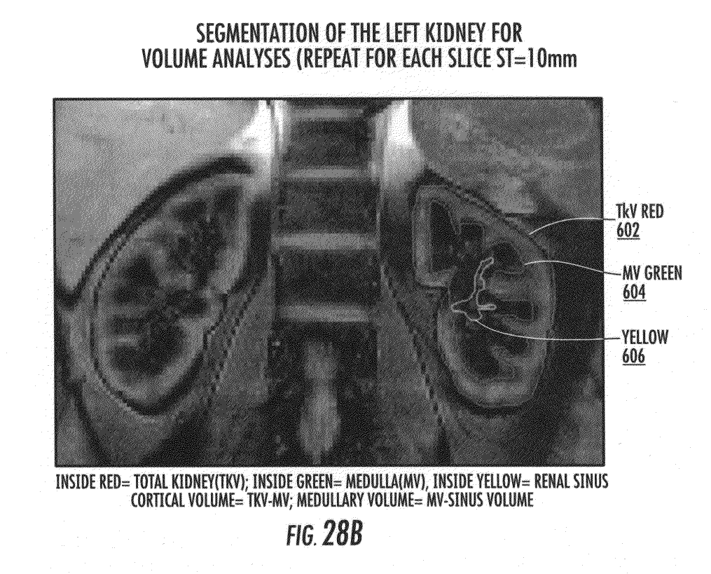

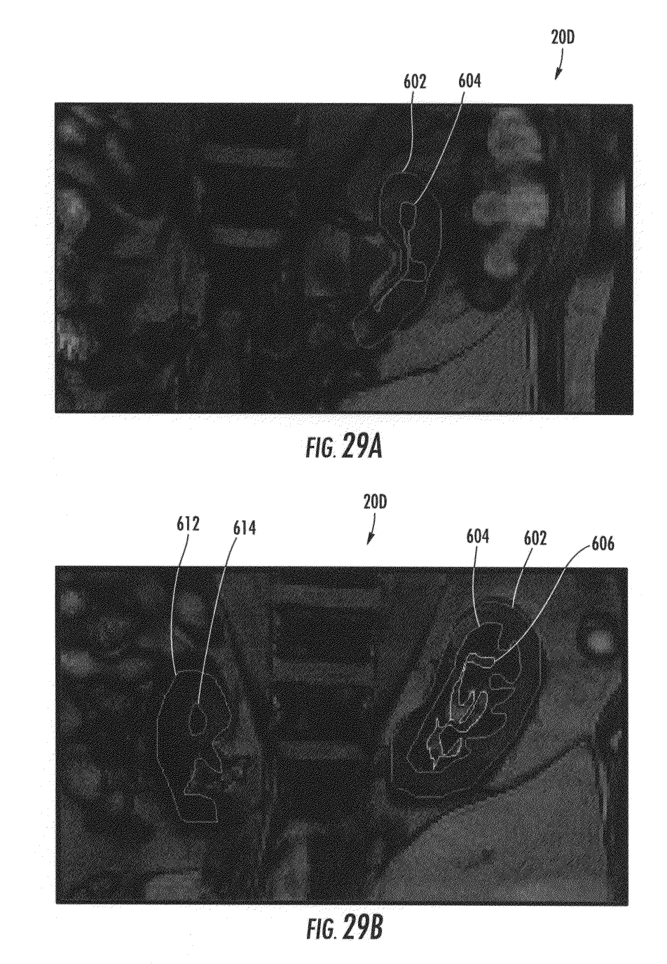

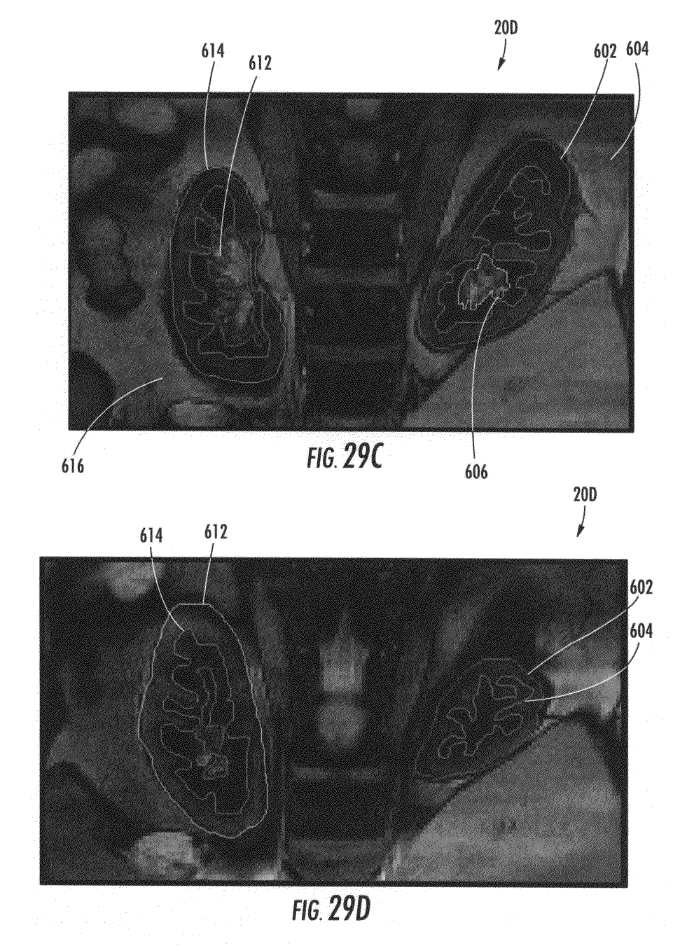

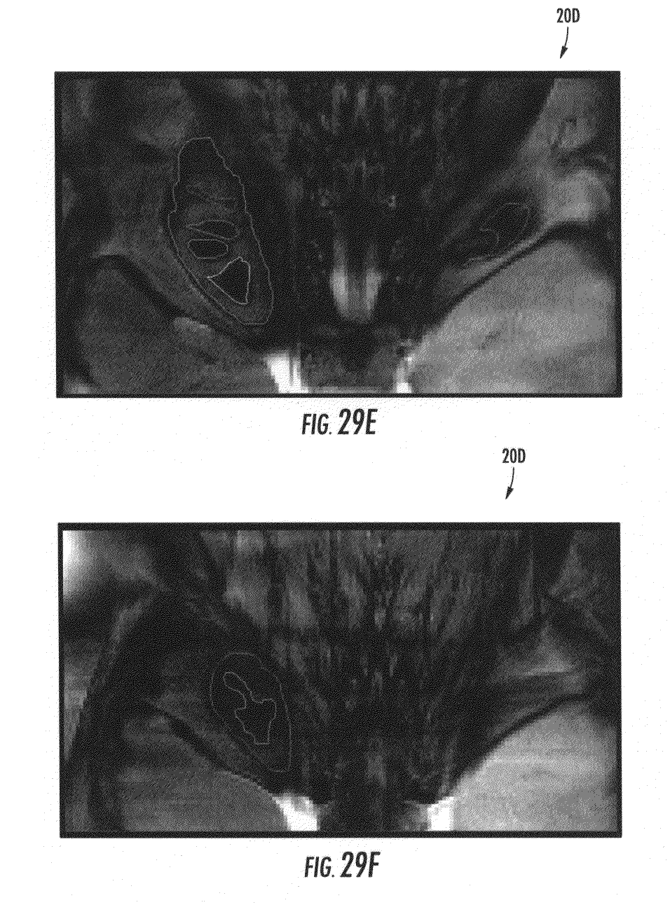

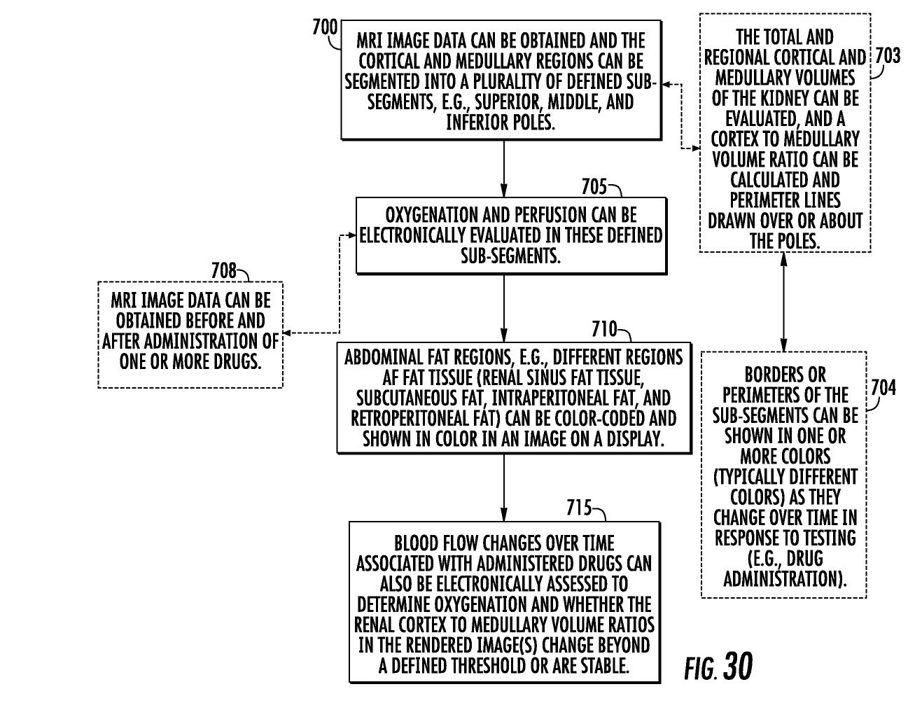

FIGS. 28A and 29A-F, illustrate that, in some embodiments, the automated system 10 can provide color enhanced or coded kidney images 600 that segment a respective patient kidney into medullary and cortical components, then quantify the volumes of these components and changes in structure or volume over time (such as pre and post drug administration). The image can be segmented or shown with a plurality of defined, color-differentiated sub-segments, e.g., superior, middle, and inferior poles. The images 600 can be generated using a 3-dimensional MRI volume acquisition of the kidney and evaluating image intensity and/or other image parameter techniques to identify the kidney volume and the components of that volume that represent the cortex, the medulla, and the hilar regions (602, 604, 606 in one kidney and 612, 614, 616 in the other). The automated system 10 can render these different components of the kidney with different colors or different color borders (perimeters) for ease in visual differentiation in an image.

FIG. 28A shows a screen display 20D with overlapping panels of segmented image slices a patient's kidney or kidneys taken over time. FIG. 28B is an example of a segmentation of a kidney for volume analyses with borders in different colors representing different kidney volumes that can be repeated for each slice (an exemplary slice thickness ST of 10 mm). The slice thicknesses can be any suitable thickness, typically between about 3 mm to about 20 mm, shown as 10 mm. The outer perimeter (red) line 602 is associated with a total kidney volume (TKV) that is inside this perimeter. The middle perimeter line (green) surrounds the medulla volume (MV) which is inside this line 604. The insidemost perimeter line 606 (yellow) is associated with a renal sinus volume (RS) which is inside this innermost perimeter. The cortical volume can be calculated as =TKV_MV; the medullary volume can be calculated as =MV-RS (renal sinus) volume.

FIGS. 29A-F show the changes in perimeter lines of the different kidney segments showing changes in volume over time (which may be due to pre or post challenge or drug therapy) for ease in clinician review (and/or automated analysis).

FIG. 30 is a flow diagram of an automated renal evaluation system 10. The kidney can segmented for volume analyses (and can be repeated for a number of slices, each slice having the same or a different slice thickness ST). For example, cortical and medullary regions can be segmented into a plurality of defined sub-segments, e.g., superior, middle, and inferior poles (block 700). Oxygenation and perfusion can be electronically evaluated in these defined sub-segments (block 705). The total and regional cortical and medullary volumes of the kidney can be evaluated, and a cortex to medullary volume ratio can be calculated and perimeter lines drawn over or about the poles (block 703). Borders or perimeters of the sub-segments can be shown in one or more colors (typically different colors) as they change over time in response to testing (e.g., drug administration) (block 704).

Abdominal fat regions, e.g., different regions of fat tissue (RS, SC, IP, RP) can be color-coded and shown in an image on a display (block 710). It is believed that RS fat is an independent predictor of severity of hypertension. See, Hypertension, 2010: 56(5): 901-6. In any event, one or more of these fat regions may provide important clinical information, particularly in conjunction with the other renal data/images.

Medications, agents or other drugs can be infused (1 or more) and perfusion and oxygenation can again be evaluated in these sub-segments (block 708). The total and regional cortical and medullary volumes of the kidney can be evaluated.

The system can also evaluate other structures such as the immediate (adjacent) surrounding structures that may influence changes in the perfusion and/or oxygenation rates, such as the perirenal and perihilar fat volumes.

The efficacy of manipulations of blood flow can also be automatically electronically assessed to determine oxygenation and whether these values preserve renal cortex to medullary volume ratios. That is, blood flow changes over time associated with administered drugs can be electronically assessed to determine oxygenation and whether the renal cortex to medullary volume ratios change beyond a defined range or value or are substantially stable (block 715).

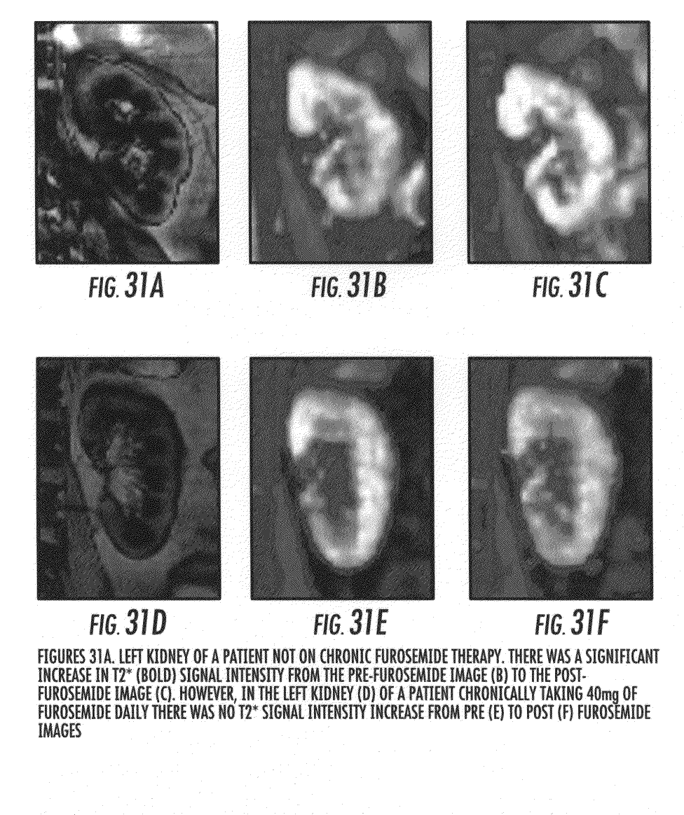

FIGS. 31A-C are images of a left kidney of a patient not on chronic furosemide therapy. FIGS. 31B and 31C illustrate a significant increase in T2* (BOLD) signal intensity from the pre-furosemide image (FIG. 31B) to the post-furosemide image (FIG. 31C). FIGS. 31D-F are images of a left kidney of a patient chronically taking 40 mg of furosemide daily. There was no T2* (BOLD) signal intensity increase from pre (FIG. 31E) to post (FIG. 31F) furosemide images.

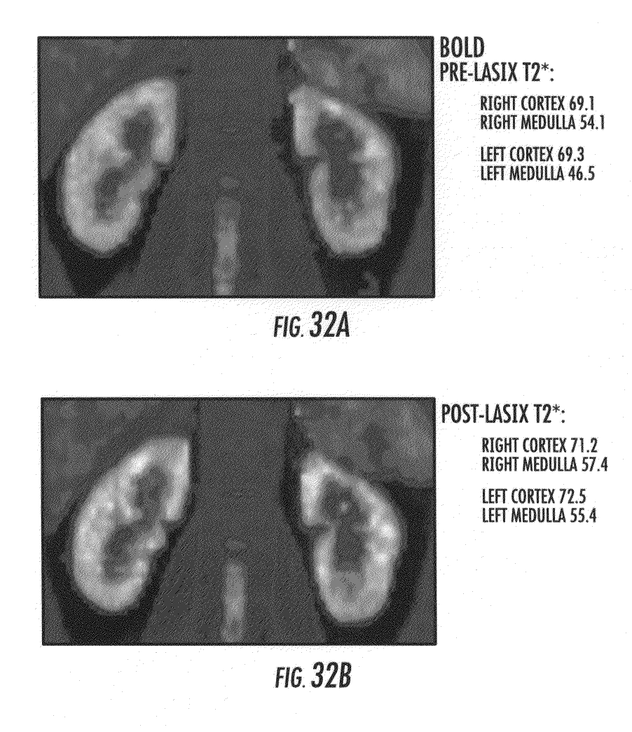

FIGS. 32A and 32B are BOLD pre-Lasix T2* and Post-Lasix T2* images of kidneys of a patient with adjacent right cortex and medulla values (pre and post). Comparisons of these values for each compartment can provide clinically important data.

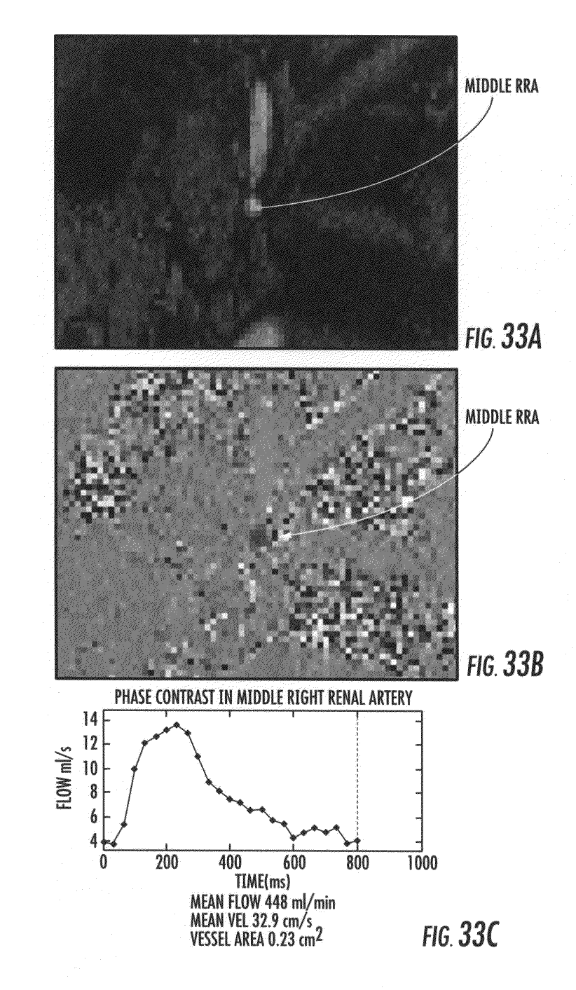

FIGS. 33A-33B are phase contrast images of the middle right renal artery (mRRA) shown with a color enhanced border (the inner red circle). FIG. 33C is a graph of flow (ml/s) per time (ms) with a mean flow of 448 ml/min, a mean velocity of 32.9 cm/s and a vessel area of 0.23 cm². These data values can be automatically calculated in some embodiments of the present invention.

In summary, the automated renal evaluation systems, modules and workstations can be highly informative and guide not only surgical interventions as well as medical interventions that preserve kidney function and prevent or delay the initiation of dialysis. Embodiments of the invention allows for one or more of: automated determination of renal viability, correlation of renal viability according to specific therapies, rapid responses and assessments of viability after short term therapies (IV, oral medications, exercise) and clinical information to clinicians to allow them to tailor therapies to preserve kidney function.

### Prophetic Examples

### I. Renal Response Screening to Drugs for Non-Renal Conditions for Therapy Selection

Many patients have diseases or conditions that require a drug therapy. Oftentimes there are several, if not many, different available drugs to treat that condition, some of which may invoke an undesirable or negative response or reaction in a kidney, while others may actually improve kidney function (e.g., perfusion, oxygenation and/or renal blood flow). The renal screening using test doses of different agents with renal evaluations using MRI image data can allow for improved treatment decisions. The renal screening for a suitable therapy for a particular patient, such as a diabetic patient or a patient with high blood pressure having impaired kidney function, may avoid increased kidney damage that might lead to dialysis.

### II. Renal Response Screening for Renal Therapies

In some embodiments, an automated renal screening with test doses can be used to facilitate shorter hospital stays and/or better outcomes for patients presenting with severely impaired kidney function resulting in hospitalization for treatment. Thus, embodiments of the invention can be used as a rapid screen using test doses and MRI image data of renal function can provide better clinical choices to identify a drug therapy that will improve or even “jump” start a kidney after a trauma, injury or acute or chronic disease, typically resulting in a hospital admission.

### III. Renal Assessment for Surgical or Medical Intervention to Delay or Prevent Dialysis

The automated systems can evaluate images of a patient to determine one or more renal tissue characteristic of the images. The characteristic of the images may, for example, be an average intensity of pixels/voxels in the region of interest. The characteristic of the pixels/voxels that is evaluated may include intensity, color, saturation and/or other characteristics of individual pixels/voxels as well as relative characteristics of multiple pixels/voxels, such as ratios, differences of pixel or voxel values between two or more images, and the like. The results of this evaluation can be automatically, electronically generated and may be provided to a user in a report format electronically on a display or in other suitable (e.g., print form) or may be provided for further analysis. The results can be pattern matched to a library of patterns that are characteristic of particular kidney injuries, diseases and/or conditions or that can predict positive or negative outcomes of one or more defined therapy alternatives, such as whether the patient is a good candidate for surgical intervention or a particular drug therapy.

### IV. Renal Assessment to Determine if Viable Candidate for Surgical Intervention

The systems can be configured to automatically identify whether a patient is likely to benefit from Renal Artery Revascularization (RA-RV) surgical intervention by electronic evaluation of MRI image data using tissue maps, such as, but not limited to, T1 and T2* tissue maps of a kidney of a patient. The systems can segment the cortical and medullay regions, assess oxygenation and perfusion in these regions, then one or more agents can be administered to the patient and, perfusion and oxygenation can be reassessed in each of these regions.

The systems can evaluate structure adjacent the kidney such as different abdominal fat volumes (e.g., perirenal and perihilar fat volumes) that can influence perfusion and/or oxygenation.

The systems can assess the efficacy of manipulations of blood flow (based on one or more administered drug or agent) to determine oxygenation and whether oxygenation values indicate renal cortex to medullary volume ratios are substantially constant (preserved) or unduly and/or negatively change.

### V. Renal Evaluations

In some embodiments, the system can be a post-data acquisition system that reviews image data of the kidney and generates (i) color coded images of abdominal fat with different fat regions/tissue shown in different color and (ii) segmented kidney images showing cortical and medullary regions in sub-segments of superior, middle and inferior middle poles with borders in different colors.

### VI. Color Spectrum Renal Maps

The renal evaluation systems can be configured to generate maps or computed images (from MRI image data) that can be presented in a heated spectrum color map or other color-coded map. Cortical and medullary ROIs can be manually or electronically automatically identified. The maps can be generated using MR images can be acquired at multiple TEs; the T2* decay curve (exponential function modeling the T2* process) can be fit on a pixel by pixel basis for the images at different times. The fitted T2* data can be extracted to generate a parametric T2* map. Pre and post furosemide (or other diuretic agent) scans can be registered and difference maps generated. The cortex and medulla regions of interest (ROIs) can be segmented electronically. The system may include a GUI input that allows a user to manually trace the regions. Smaller ROIs can also be used to compare values in different regions of the kidney.

### VII. Automated Renal Evaluation Systems

The automated systems can provide perfusion information that can be combined with one or more other measures of function or physiology in a color-coded representation (tissue map) of the kidney where the color coding can indicate tissue viability.

The images can include each or combinations of image data from two or more of T1, T2 or T2* renal images. Stress ratios of one or more of the different tissue maps can be electronically generated.

### VIII. Automated Renal Evaluation Systems

A structural angiogram can be provided as a 3D set of data with the ability to zoom, rotate, slice and reformat. Software (electronic) calipers can be provided to measure lumen diameter or area at points along a renal artery for quantification of renal stenosis severity.

Embodiments of the invention can automatically identify those patients having severe stenosis, e.g., about 75% or greater occlusion.

Flow measurements can be automatically determined using images where pixel values reflect velocity of blood flow in the renal artery.

The measurements can be automated using a circuit such as a computer program or software for automatic lumen segmentation and extraction of parameters of interest such as mean flow over a cardiac cycle, peak velocity and flow volume. Ratios before and after drug or agent administration may be used to provide flow reserve measures which indicate vascular functional reserve.

Selected absolute or relative values of each pixel in regions of interest in one or more images can be evaluated, e.g., electronically evaluated to determine the value for each pixel correlated to a respective location.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims.

That which is claimed is:

1. A renal evaluation system, comprising:
a circuit comprising at least one processor configured to: (i) segment cortical and medullary regions of different MRI kidney image slices of a respective patient into defined sub-segments for volume analysis and associate borders of the defined sub-segments with a respective color; (ii) assess oxygenation and perfusion in the defined sub-segments before and after one or more agents are administered to a respective patient; and (iii) generate a color coded image of abdominal fat adjacent a respective kidney of a patient; and
at least one display in communication with the circuit configured to display the color coded image of abdominal fat of a patient and at least one image slice of a segmented kidney with defined sub-segments with color borders.

2. The system of claim 1, wherein the defined sub-segments include a total kidney volume, a medulla volume, and a renal sinus volume, and wherein the circuit is configured to analyze each kidney image slice having a slice thickness between about 3 mm to about 20 mm, to calculate a cortical volume as equal to total kidney volume minus medulla volume and to calculate a medullary volume as equal to the medulla volume minus the renal sinus volume, and wherein the circuit is configured to evaluate whether blood flow changes in response to administered agents preserve or alter renal cortex to medullary volume ratios.

3. The system of claim 1, wherein the circuit is configured to calculate blood flow and percent stenosis of at least one renal artery.

4. The system of claim 1, wherein the circuit is configured to identify whether the patient is likely to benefit or likely not to benefit from a medical or procedural therapy.

5. The system of claim 1, wherein the circuit is configured to analyze at least one of tissue oxygenation, vascular oxygenation, renal arterial blood flow by comparing base line MRI image data and MRI images obtained after administration of a therapy delivered proximate in time to an MRI scan session used to obtain post-therapy MRI image data of the kidney or kidneys.

6. The system of claim 1, wherein the circuit is further configured to generate color and/or heat spectrum tissue maps of a patient's kidney or kidneys, the tissue maps illustrating the kidney or kidneys with associated pixel values defined based at least in part on at least one of (i) a ratio of T1 and T2*; (ii) a weighted combination of T1 and T2*, or (iii) a T2* difference map and a T1 difference map using corresponding pixels associated with respective T1 and T2* MR images obtained before and after administration of an agent, the T2* difference map visually illustrates vascular oxygenation in color scale and the T1 difference map visually illustrates tissue oxygenation in color scale.

7. A clinician workstation, comprising:
a circuit configured to generate tissue maps of a patient's kidney or kidneys, the tissue maps illustrating the kidney or kidneys with associated pixel values defined based at least in part on at least one of (i) a ratio of T1 and T2*; (ii) a weighted combination of T1 and T2*, or (iii) a T2* difference map and a T1 difference map using corresponding pixels associated with respective T1 and T2* MR images obtained before and after administration of an agent, the T2* difference map visually illustrates vascular oxygenation in color scale and the T1 difference map visually illustrates tissue oxygenation in color scale; and
at least one display in communication with the circuit configured to display the generated tissue maps.

8. The workstation of claim 7, wherein the T1 and T2* MR image data includes T1 and T2* image data taken before and after a drug challenge, and wherein the T1 and T2* image data is obtained with a non-contrast agent MRI pulse sequence.

9. A circuit with at least one processor configured to generate color-coded renal tissue maps showing renal vascular oxygenation and tissue oxygenation using image data from a T2*difference map and image data from a T1 difference map, the difference maps calculated by subtracting a defined parameter of pixels of MRI images taken from pre and post-drug or agent administration.

10. A renal evaluation signal processor circuit, comprising:
a renal image processing module configured to automatically (i) generate at least one heated spectrum color map of one or both kidneys of a patient using T1 and T2* MRI image data; (ii) calculate blood flow measurements of renal arteries; and (iii) quantify occlusion and/or stenosis of at least one renal artery.

11. The circuit of claim 10, wherein the renal image processing module is in communication with a circuit configured to evaluate whether the patient is likely to benefit from revascularization surgery.

12. A method of evaluating whether a patient is likely to benefit from Renal Artery Revascularization (RA-RV) surgical intervention comprising:
electronically evaluating T1 and T2*difference tissue maps of a kidney of a patient;
electronically defining a degree of stenosis in at least one renal artery;
electronically calculating renal artery blood flow rate;
electronically color coding different abdominal fat compartments in MRI image slices of the kidney; and
displaying the color coded fat compartments, the degree of stenosis and the calculated renal artery blood flow rate.

13. A computer program product for evaluating renal function in a patient, comprising:
a non-transitory computer readable medium having computer readable program code embodied therein, the computer readable program code comprising:
computer readable program code configured to generate at least one color-coded renal tissue map using MRI image slices of a kidney of a patient; and
computer readable program code configured to determine a likelihood of a patient to respond favorably to revascularization therapy.

14. A computer program product according to claim 13, wherein at least some of the MR image slices are obtained after administration of a diuretic to the patient.

15. A therapeutic renal screening system, comprising:
a circuit configured to electronically analyze MRI images of at least one kidney of a subject to evaluate renal function based on renal responses to test doses of each of a plurality of different defined therapeutic agents, wherein the circuit evaluates at least one of (a) tissue oxygenation, (b) vascular oxygenation, and (c) renal artery blood flow rates to evaluate the renal responses, and wherein the circuit generates a renal risk report for the different therapeutic agents based on the patient's renal response to the test doses of each of the agents.

16. The system of claim 15, wherein the different agents are for treating a condition other than kidney disease.

17. The system of claim 15, further comprising a workstation with a display in communication with the circuit, the circuit configured to analyze the MRI images, generate the renal risk report and transmit the renal risk report to the workstation display within about 24 hours after a respective subject's MR scan session used to obtain the MRI images.

18. The system of claim 16, wherein the circuit is configured to generate a rapid screening analysis with one or more associated reports, the analysis being carried out and the one or more reports transmitted to a clinician within about 2 hours after the subject's MR scan session.

19. The system of claim 15, wherein the circuit is in communication with an infusion pump, a plurality of test doses of the different therapeutic agents configured for IV administration and a control circuit for directing the serial delivery of the test doses, wherein the therapeutic agents are administered as oral agents during therapeutic use, and wherein the test doses are substantially pharmaceutically equivalent formulations of the therapeutic agents configured for IV administration.

20. The system of claim 15, further comprising:
a display in communication with the circuit; and
an electronic library module in communication with the circuit, the electronic library module comprising lists of different therapeutic agents correlated to different defined conditions, and wherein a user can select a condition from the defined conditions and the circuit presents associated different therapeutic agents to the display.

21. The system of claim 20, wherein the library of different conditions include at least two of the following conditions: diabetes, COPD, asthma, heart failure, heart disease, chemotherapy, infection, and high blood pressure.

22. The system of claim 15, wherein the test doses are provided in a kit of test vials or pouches.

23. The system of claim 15, wherein the risk report includes a color risk evaluation for each of the different therapeutic agents ranging from high to low risk of kidney complications or undesired kidney response, including a first color for low risk, a second color for a moderate risk, and third color for a high risk.

24. The system of claim 23, wherein the risk report includes a numerical risk index evaluation for each of the different therapeutic agents ranging from high to low risk of kidney complications or undesired kidney response, on a numerical index from 1-10, with 1 being a low risk and 10 being a high risk.

25. The system of claim 15, wherein the risk report includes a color risk evaluation as well as a numerical risk index from 1-10 for each of the different therapeutic agents ranging from high to low risk of kidney complications or undesired kidney response, including “green” and a number “1” for low risk, “yellow” and a number “5” for a moderate risk, and “red” and a number “10” for a high risk on a numerical index from 1-10.

26. A method of screening patients to inhibit potential renal complications associated with a drug therapy, comprising:
providing a plurality of test doses of different drugs suitable for treating a defined condition;
serially intravenously administering the test doses of the different drugs to a patient while the patient is in a high-field magnet of an MRI Scanner;
electronically obtaining MRI image data of the patient associated with each administered drug; and
electronically analyzing the MRI image data to predict whether the patient is likely to have a risk of renal injury, renal dysfunction or renal damage for each of the administered drugs.

27. The method of claim 26, further comprising generating a risk report that summarizes a predicted risk for each of the administered drugs based on the analyzed MRI image data.

28. The method of claim 26, wherein the electronically analyzing the MRI image data is carried out within about 24 hours of a respective patient MRI scan session.

29. The method of claim 26, wherein the defined condition is one of diabetes, COPD, asthma, heart failure, heart disease, chemotherapy, infection, and high blood pressure.

30. The method of claim 29, wherein the defined condition is high blood pressure.

31. The method of claim 26, wherein the electronically analyzing determines a measure of blood flow in a renal artery, and a pattern of oxygenation and perfusion for each of the administered agents.

32. A method of selecting a drug therapy for improving renal function, comprising:
serially intravenously administering the test doses of the different drugs to a patient while the patient is in a high-field magnet of an MRI Scanner;
electronically obtaining MRI image data of the patient associated with each administered drug;
electronically analyzing the MRI image data to predict whether the patient is likely to respond favorably or not to a respective administered drug; and
electronically generating a rapid evaluation report with a summary of favorable or unfavorable renal response for each of the administered drugs.

33. A method of analyzing renal function comprising:
electronically obtaining MRI image data of at least one patient kidney;
electronically segmenting cortical and medullary regions of the kidney into sub-segments including superior, middle and inferior poles;
electronically evaluating oxygenation and perfusion in the sub-segments;
electronically evaluating volumes of a plurality of different abdominal fat subvolumes adjacent the at least one kidney; and
electronically evaluating whether blood flow changes in response to administered agents preserve or alter renal cortex to medullary volume ratios.

34. The method of claim 33, further comprising displaying a color coded axial slice image of abdominal fat surrounding a kidney including different fat volumes shown in different colors, the different fat volumes including renal sinus fat, retroperitoneal fat, subcutaneous fat and intraperitoneal fat.

35. The method of claim 33, further comprising showing the sub-segments with different color borders.
